(19) United States
(12) Patent Application Publication
(10) Pub. No.: US 2022/0305005 A1
(43) Pub. Date: Sep. 29, 2022

(54) METAL CHELATOR COMBINATION THERAPY FOR THE TREATMENT OF CANCER

(71) Applicant: Board of Regents, The University of Texas System, Austin, TX (US)

(72) Inventor: Maro OHANIAN, Houston, TX (US)

(21) Appl. No.: 17/310,262

(22) PCT Filed: Jan. 28, 2020
(86) PCT No.: PCT/US2020/015407

§ 371 Date: Jul. 27, 2021

Related U.S. Application Data
(60) Provisional application No. 62/797,752, filed on Jan. 28, 2019.

(51) Int. Cl. A61K 31/496; A61K 33/30; A61K 33/04; A61K 33/06; A61K 33/24; A61K 31/375; A61K 31/4196; A61K 31/4412; A61K 31/16; A61K 31/132; A61K 47/68; A61K 31/7076; A61K 31/704; A61K 31/7068; A61K 31/353; A61P 35/02; A61K 31/198; A61K 31/194

(52) U.S. Cl. CPC A61K 31/496; A61K 33/30; A61K 33/04; A61K 33/06; A61K 33/24; A61K 31/375; A61K 31/4196; A61K 31/4412; A61K 31/16; A61K 31/132; A61K 47/6849; A61K 31/7076; A61K 31/704; A61K 31/7068; A61K 31/353; A61P 35/02; A61K 31/198; A61K 31/194

(57) ABSTRACT

Provided are methods for the treatment of cancers including leukemias, that utilize combination therapies. In some embodiments, a metal chelator is administered to a patient in combination with a cancer therapy, zinc, selenium, magnesium, and vitamin C to treat the cancer. In some embodiments, increased efficacy of the cancer therapy can be observed, and/or lower dosages of a chemotherapeutic may be administered to the subject as a result of the combination therapy.

### BACKGROUND OF THE INVENTION

This application claims the benefit of U.S. Provisional Application No. 62/797,752, filed 28 Jan. 2019, the contents of which are hereby incorporated by reference as if written herein in its entirety.

### 1. FIELD OF THE INVENTION

The present invention relates generally to the field of molecular biology and medicine. More particularly, it concerns compositions and methods for the treatment of cancer.

### 2. DESCRIPTION OF RELATED ART

There are currently no standard treatment protocols for broad-spectrum metal detoxification or metal rebalancing for cancer, cancer maintenance therapy, cancer prevention, or for treatment or prevention of acute and chronic diseases. Cancer continues to be a significant clinical problem. There is a need for new and improved methods for the treatment of cancers and risk assessment.

### SUMMARY OF THE INVENTION

The present invention is based, in part, on the observation that various combination therapies that include metal chelators may be particularly beneficial for the treatment of various cancers including, e.g., leukemias. In some embodiments, one, two, or more chelators are administered to a mammalian subject (e.g., a human patient) with a cancer to selectively bind one or more metals, such as copper (Cu), arsenic (As), cesium (Cs), and/or lead (Pb) in the subject.

In one embodiment, there is provided a method of treating a disease, preferably a cancer, in a mammalian subject comprising administering to the subject therapeutically effective amounts of (i) a metal chelator; (ii) a cancer therapy, wherein the cancer therapy is a chemotherapy, an epigenetic therapy, an immunotherapy, or a targeted cancer therapy; and (iii) at least one of, more preferably at least two of, more preferably at least three of, and even more preferably all of zinc, selenium, magnesium, and/or vitamin C. It is anticipated that, in some embodiments, vitamin C can be replaced with another antioxidant or reducing agent such as, e.g., amifostine. In some embodiments, multiple chelators may be used. If the chelator is dexrazoxane or another chelator, then the method may comprise administering a second metal chelator to the subject. In some embodiments, one or more metal chelator is administered to the subject, wherein the one or metal chelators is not dexrazoxane. In specific aspects, the subject is a human.

In some aspects, the cancer is a leukemia or a hematological malignancy. In certain aspects, the leukemia is acute myeloid leukemia (AML), acute lymphoblastic leukemia (ALL), chronic lymphocytic leukemia (CLL), or chronic myeloid leukemia (CML). In particular aspects, the subject has myelodysplastic syndrome (MDS). In some aspects, the cancer has relapsed or is refractory to a previous treatment.

In certain aspects, the metal chelator is a dithiol chelator. In some aspects, the dithiol chelator is dimercaptosuccinic acid (DMSA), 2,3-dimercapto-1-propanesulfonic acid (DMPS), or dimercaprol (BAL). In some embodiments, the chelator is dimercaptosuccinic acid (DMSA), 2,3-dimercapto-1-propanesulfonic acid (DMPS), N-(2,3-dimercaptopropyl)-phthalamidic acid (DMPA), pentetate calcium trisodium (Ca-DTPA), or dimercaprol (BAL). In some embodiments, the method comprises administering both dimercaptosuccinic acid (DMSA) and 2,3-dimercapto-1-propanesulfonic acid (DMPS) to the subject. In some embodiments, the chelator is gadolinium chelator, such as a bifunctional gadolinium(III) chelator. In specific aspects, the metal chelator is calcium-EDTA (Ca-EDTA) or calcium disodium EDTA. In some aspects, the metal chelator is an iron chelator. In certain aspects, the metal chelator is deferasirox, deferiprone, or deferoxamine In some aspects, the metal chelator is N-acetyl-cysteine (NAC). In some aspects, the metal chelator is a copper chelator. In certain aspects, the copper chelator is trientine or tetrathiomolybdate. In specific aspects, the metal chelator is dexrazoxane. In some embodiments, the chelator is a metal-chelator complex.

In additional aspects, the method further comprises administering to the subject a therapeutically effective dose of dexrazoxane. In some aspects, the method further comprises administering to the subject a therapeutically effective dose of amifostine. In some embodiments, the method comprises administering one, two, three, or all of zinc, selenium, magnesium, and vitamin C to the subject. In some embodiments, the method comprises administering zinc, selenium, magnesium, and vitamin C to the subject.

In some aspects, the cancer therapy is a chemotherapy. In particular aspects, the chemotherapy is chosen from one or more of mylotarg, cladribine, idarubicin, cytarabine, and CPX-351. In particular aspects, the chemotherapy is mylotarg, cladribine, idarubicin, or cytarabine. In specific aspects, the chemotherapy is mylotarg, cladribine, idarubicin, and cytarabine (CLIA-M). In specific aspects, the chemotherapy is mylotarg, cladribine, idarubicin, cytarabine (CLIA-M), and CPX-351.

In further aspects, the method further comprises administering Amifostine to the subject.

In some embodiments, the cancer is a leukemia, preferably AML; wherein the cancer therapy is a chemotherapy; and wherein all of zinc, selenium, magnesium, and vitamin C are administered to the subject. It is anticipated that, in some embodiments, vitamin C can be replaced with another antioxidant or reducing agent such as, e.g., amifostine.

In particular aspects, the chemotherapy is an anthracycline, a Berlin-Frankfurt-Munster (BFM) chemotherapy, a hyper-CVAD chemotherapy comprising (cyclophosphamide, vincristine, doxorubicin, and dexamethasone), a hypomethylating therapy (e.g., decitabine or azacytidine), cytarabine, clofarabine, or cladribine.

In some aspects, the cancer therapy is an immunotherapy. In certain aspects, the immunotherapy is a monoclonal antibody or an immune checkpoint inhibitor, rituximab, ofatumumab, or blinatumomab, inotuzumab, gemtuzumab ozogamicin, nivolumab, ipilumumab, or an immune checkpoint inhibitor targeting PD-1/PD-L1. In certain aspects, the cancer therapy is a targeted therapy. In some aspects, the targeted therapy is a FLT3 inhibitor, a BCR-ABL tyrosine kinase inhibitor, a JAK-2 inhibitor, an IDH1 or IDH2 inhibitor, or gemtuzumab ozogamicin. In some aspects, the targeted therapy is a chosen from a FLT3 inhibitor, a BCR-ABL tyrosine kinase inhibitor, a JAK-2 inhibitor, an IDH1 or IDH2 inhibitor, gemtuzumab ozogamicin, a BCL2-inhibitor or BCL-2 targeted therapy, a RAS/MEK inhibitor, a CDK inhibitor, glasdegib and/or another inhibitor of sonic hedgehog. In some preferred embodiments, the subject is a human In some embodiments, the method results in the reduction or clearance of one or more mutations or cytogenetic abnormalities in the cancer. In some embodiments, the cancer therapy is an epigenetic therapy or a targeted therapy.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Lower magnesium at a level less than 33049.03 ppb was associated with inferior survival in patients with AML.

FIG. 2: Excess copper at a level ≥1130.69 ppb was associated with inferior survival in patients with AML.

FIG. 3: Lower zinc (Zn) under 546.02 ppb was associated with inferior survival in AML.

FIG. 4: Higher arsenic (As) at a level at a level ≥1.06 ppb was associated with inferior survival in AML.

FIG. 5: Higher antimony (Sb) at a level ≥0.57 ppb was associated with inferior survival in AML.

FIG. 6: Higher cesium (Cs) at a level ≥0.71 ppb was associated with inferior survival in AML.

FIG. 7: Higher lead (Pb) at a level ≥0.91 ppb was associated with inferior survival in AML.

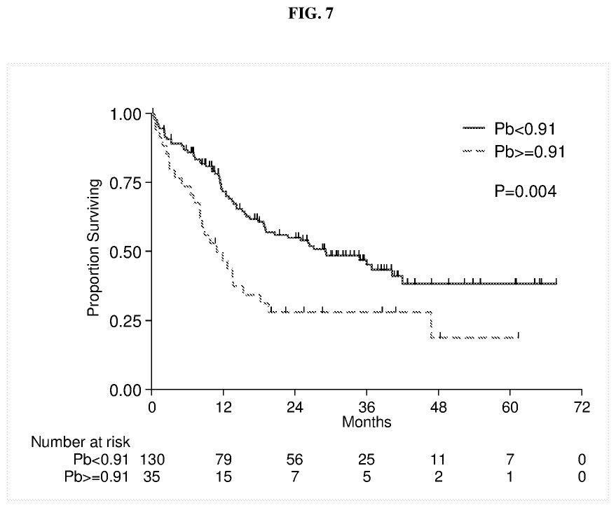

FIG. 8: Kaplan-Meier curves comparing overall survival in AML patients with high (≥2.43 mmol/L) and low (<2.43 mmol/L) serum calcium values.

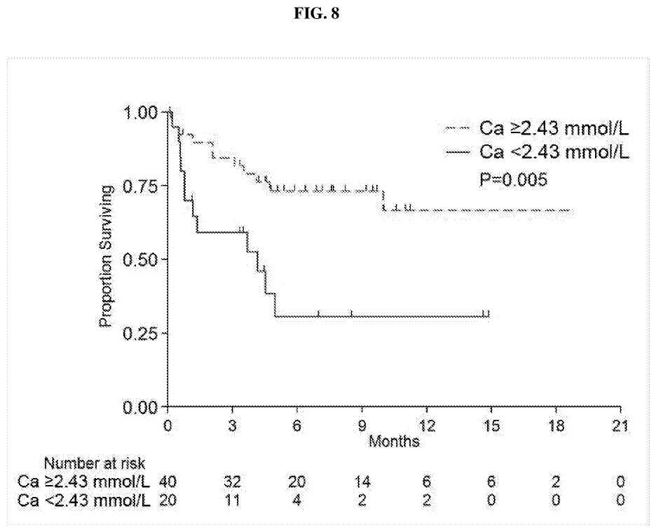

FIG. 9: Kaplan-Meier curves comparing overall survival in AML patients with high (≥0.750 nmol/L) and low (<0.750 nmol/L) serum cadmium values.

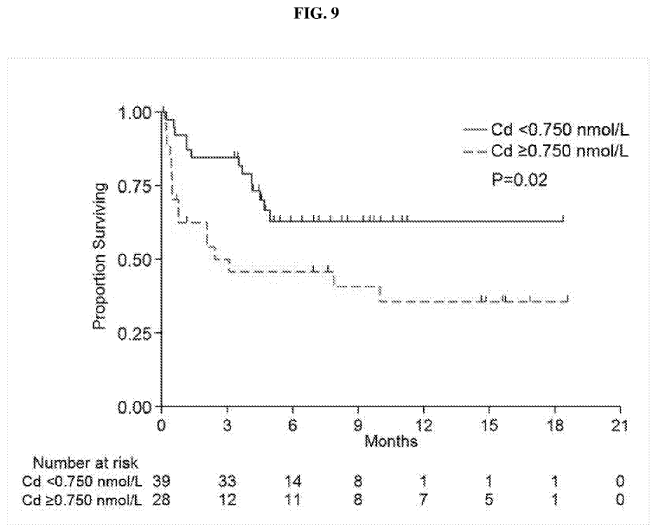

FIG. 10: Kaplan-Meier curves comparing overall survival in AML patients with high (≥0.692 μmol/L) and low (<0.692 μmol/L) serum selenium values.

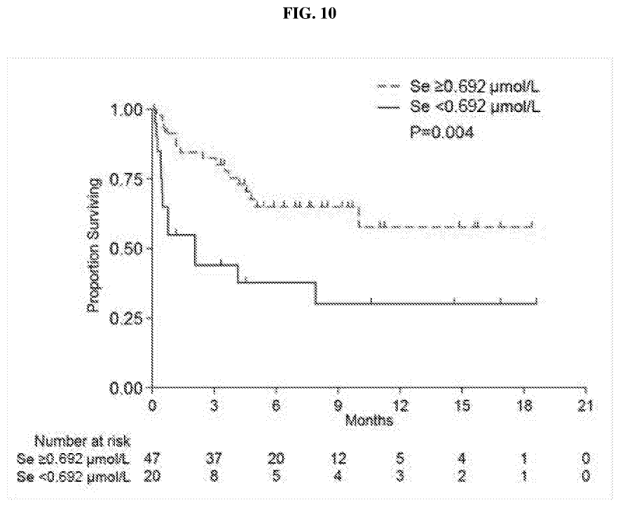

FIG. 11: Overall survival by groupings of metal scores is shown for patients treated in the study of Example 2. Survival was significantly worse in patients with higher metal scores.

FIG. 12: Overall survival by groupings of metal scores is shown for patients treated in the second study of Example 2 done at a different hospital. Survival was significantly worse in patients with higher metal scores.

### DESCRIPTION OF ILLUSTRATIVE EMBODIMENTS

The instant application overcomes limitations in the prior art by providing, in some aspects, methods and compositions for the treatment of cancers. In some aspects, compositions and methods protocol for the treatment and/or maintenance of cancer, leukemia, or other hematologic malignancies with a combination of (i) one or more metal chelators with (ii) one or more antioxidants, vitamins, or minerals, and (iii) a chemotherapy, epigenetic therapy, immunotherapy, or targeted therapy.

In certain embodiments, the present disclosure provides a new treatment comprising broad-spectrum detoxification of toxic and/or essential metals while replacing essential minerals and antioxidants during cancer treatment and prevention. This novel approach is justified by clinical laboratory data demonstrating significantly elevated levels of toxic metals and imbalances and deficiencies of essential metals, as well as favorable clinical outcomes in AML/MDS patients receiving metal chelators and antioxidants/minerals during AML/MDS treatment. Further provided herein are combinations that can be individually tailored to a patient's specific metal abnormalities, depending on specific metal derangements.

Further embodiments of the invention are described below.

Embodiment 1. Provided herein is:
a method of treating a disease in a mammalian subject; or
a method of reducing the levels of one or more toxic metals to undetectable levels in the serum and/or bone marrow of a subject with cancer;
comprising administering to the subject:
   (i) one or more metal chelators;
   (ii) at least one antioxidant, vitamin, or mineral (preferably an essential mineral); and
   (iii) optionally, at least one anti-cancer therapy,
   in a therapeutically effective amount.

Embodiment 2. The method of Embodiment 1, wherein the method additionally comprises administering to the subject an anti-cancer therapy.

Embodiment 3. Also provided herein is:
a method of treating cancer in a mammalian subject; or
a method of reducing the levels of one or more toxic metals to undetectable levels in the serum and/or bone marrow of a subject with cancer;
comprising administering to the subject:
   (i) one or more metal chelators;
   (ii) at least one anti-cancer therapy; and
   (iii) optionally, at least one antioxidant, vitamin, or mineral (preferably an essential mineral);
   in a therapeutically effective amount.

Embodiment 4. The method of Embodiment 3, wherein the method additionally comprises administering to the subject at least one antioxidant, vitamin, and/or an mineral (preferably an essential mineral).

Embodiment 5. The method of any of Embodiments 1-4, wherein the one or more metal chelators are broad-spectrum metal chelators; for example wherein at least one of the one or more metal chelators is/are capable of chelating at least two metals, and/or wherein the one or more metal chelators is/are capable of chelating potentially toxic/non-essential metal(s).

Embodiment 6. The method of Embodiment 5, wherein the one or more metal chelators is/are administered in an amount effective to reduce the levels of the at least two metals and/or in an amount effective to reduce the levels of potentially toxic/non-essential metal(s).

Embodiment 7. The method of either of Embodiments 5 and 6, wherein at least two chelators are administered, and the chelators are administered concurrently or sequentially.

Embodiment 8. The method of any of Embodiments 1-7, wherein the one or more metal chelators are chosen from EDTA, dimercaptosuccinic acid (DMSA), 2,3-dimercapto-1-propanesulfonic acid (DMPS), BAL, N-acetylcysteine (NAC), deferasirox, deferiprone, deferoxamine, pentetate calcium trisodium (Ca-DPTA), pentetate zinc trisodium (Zn-DTPA), trientine, tetrathiomolybdate, and dexrazoxane.

Embodiment 9. The method of any of Embodiments 1-7, wherein the one or more metal chelators are chosen from EDTA, dimercaptosuccinic acid (DMSA), 2,3-dimercapto-1-propanesulfonic acid (DMPS), BAL, N-acetylcysteine (NAC), deferasirox, deferiprone, deferoxamine, pentetate calcium trisodium (Ca-DPTA), trientine, tetrathiomolybdate, and dexrazoxane.

Embodiment 10. The method of any of Embodiments 1-7, wherein the metal chelator is chosen from dimercaptosuccinic acid (DMSA), 2,3-dimercapto-1-propanesulfonic acid (DMPS), N-(2,3-dimercaptopropyl)-phthalamidic acid (DMPA), pentetate calcium trisodium (Ca-DTPA), and dimercaprol (BAL).

Embodiment 11. The method of any of Embodiments 1-7, wherein the metal chelator is a dithiol chelator.

Embodiment 12. The method of Embodiment 11, wherein the metal chelator is chosen from dimercaptosuccinic acid (DMSA), 2,3-dimercapto-1-propanesulfonic acid (DMPS), and N-(2,3-dimercaptopropyl)-phthalamidic acid (DMPA); or the method of Embodiment 11, wherein the metal chelator is chosen from dimercaptosuccinic acid (DMSA), 2,3-dimercapto-1-propanesulfonic acid (DMPS), N-(2,3 -dimercaptopropyl)-phthalamidic acid (DMPA), and dexrazoxane.

Embodiment 13. The method of any of Embodiments 1-7, wherein the metal chelator is an iron chelator.

Embodiment 14. The method of Embodiment 13, wherein the metal chelator is deferasirox, deferiprone, or deferoxamine

Embodiment 15. The method of any of Embodiments 1-7, wherein the metal chelator is chosen from N-acetyl-cysteine (NAC) and a gadolinium chelator, such as a bifunctional gadolinium(III) chelator.

Embodiment 16. The method of any of Embodiments 1-7, wherein the metal chelator is a copper chelator.

Embodiment 17. The method of Embodiment 16, wherein the copper chelator is trientine or tetrathiomolybdate.

Embodiment 18. The method of any of Embodiments 1-7, wherein the metal chelator is dexrazoxane.

Embodiment 19. The method of any of Embodiments 1-18, wherein the metal(s) chelated is/are chosen from arsenic (As), aluminum (Al), antimony (Sb), Barium (Ba), boron (B), cadmium (Cd), Cerium (Ce), Chromium (Cr), lead (Pb), mercury (Hg), neodymium (Nd), Nickel (Ni), selenium (Se), tin (Sn), titanium (Ti), uranium (U), and vanadium (V).

Embodiment 20. The method of any of Embodiments 1-18, wherein the metal(s) chelated is/are chosen from arsenic (As), antimony (Sb), boron (B), cadmium (Cd), lead (Pb), mercury (Hg), neodymium (Nd), tin (Sn), titanium (Ti), uranium (U), vanadium (V)

Embodiment 21. The method of any of Embodiments 1-18, wherein the metal(s) chelated is/are chosen from cadmium (Cd), lead (Pb), antimony (Sb), and arsenic (As).

Embodiment 22. The method of any of Embodiments 1-18, wherein the metal(s) chelated is/are chosen from antimony (Sb), cadmium (Cd), mercury (Hg), arsenic (As), and uranium (U).

Embodiment 23. The method of any of Embodiments 1-18, wherein the metal(s) chelated is/are chosen from mercury (Hg), antimony (Sb), and lead (Pb).

Embodiment 24. The method of any of Embodiments 19-23, wherein two or more metals are chelated.

Embodiment 25. The method of any of Embodiments 1-24, wherein the antioxidant(s), vitamin(s) or essential mineral(s) is/are chosen from zinc, selenium, magnesium, rubidium, and vitamin C; or the method of any of Embodiments 1-24, wherein the antioxidant(s), vitamin(s) or essential mineral(s) is/are chosen from zinc, selenium, magnesium, rubidium, ascorbic acid (vitamin C), α-tocopherol (vitamin E), glutathione, lipoic acid, uric acid, carotenoids (e.g., beta-carotene, lycopene), flavonoids (e.g. quercetin), retinol, ubiquinol (coenzyme Q), taurine, N-acetylcysteine (NAC), and amifostine.

Embodiment 26. The method of Embodiment 25, wherein at least one of zinc, selenium, magnesium, rubidium, and vitamin C are administered.

Embodiment 27. The method of Embodiment 25, wherein at least two of zinc, selenium, magnesium, rubidium, and vitamin C are administered.

Embodiment 28. The method of Embodiment 25, wherein at least three of zinc, selenium, magnesium, rubidium, and vitamin C are administered.

Embodiment 29. The method of Embodiment 25, wherein at least three of zinc, selenium, magnesium, and vitamin C are administered.

Embodiment 30. The method of Embodiment 25, wherein zinc, selenium, magnesium, rubidium, rubidium, and vitamin C are administered.

Embodiment 31. The method of Embodiment 25, wherein zinc, selenium, magnesium, and vitamin C are administered.

Embodiment 32. The method of any of Embodiments 1-31, wherein the disease is a proliferative disease.

Embodiment 33. The method of Embodiment 32, wherein the disease is chosen from cancer, a myeloproliferative neoplasm (MPN), myelodysplastic syndrome (MDS), bone marrow disease; bone marrow failure; and a cytopenia.

Embodiment 34. The method of Embodiment 33, wherein the disease is cancer.

Embodiment 35. The method of Embodiment 34, wherein the cancer has relapsed or is refractory to a previous treatment.

Embodiment 36. The method of Embodiment 35 wherein the cancer is a hematologic malignancy.

Embodiment 37. The method of Embodiment 36, wherein the cancer is a leukemia.

Embodiment 38. The method of Embodiment 37, wherein the leukemia is chosen from acute myeloid leukemia (AML), acute lymphoblastic leukemia (ALL), chronic lymphocytic leukemia (CLL), chronic myeloid leukemia (CML).

Embodiment 39. The method of Embodiment 38, wherein the leukemia is AML.

Embodiment 40. The method of Embodiment 33, wherein the disease is myelodysplastic syndrome (MDS).

Embodiment 41. The method of Embodiment 33, wherein the disease is a myeloproliferative neoplasm (MPN).

Embodiment 42. The method of Embodiment 33, wherein the disease is a bone marrow disease or bone marrow failure.

Embodiment 43. The method of Embodiment 33, wherein the disease is a cytopenia.

Embodiment 44. The method of Embodiment 43, wherein the cytopenia is an idiopathic cytopenia.

Embodiment 45. The method of any of Embodiments 1, 2, and 5-44, wherein the method additionally comprises administering to the subject a therapeutically effective amount of an anti-cancer therapy.

Embodiment 46. The method of any of Embodiments 1-45, wherein the anti-cancer therapy is chosen from a chemotherapy, an epigenetic therapy, an immunotherapy, or a targeted cancer therapy.

Embodiment 47. The method of Embodiment 45, wherein the anti-cancer therapy is an anti-cancer pharmacologic therapy.

Embodiment 48. The method of Embodiment 47, wherein the anti-cancer pharmacologic therapy comprises one or more agents chosen from mylotarg, cladribine, idarubicin, and cytarabine.

Embodiment 49. The method of Embodiment 47, wherein the anti-cancer pharmacologic therapy comprises mylotarg, cladribine, idarubicin, and cytarabine (“CLIA-M”).

Embodiment 50. The method of Embodiment 46 wherein the anti-cancer therapy is an immunotherapy.

Embodiment 51. The method of Embodiment 50, wherein the immunotherapy is chosen from a monoclonal antibody and an immune checkpoint inhibitor.

Embodiment 52. The method of Embodiment 50, wherein the immunotherapy is chosen from rituximab, ofatumumab, or blinatumomab, inotuzumab, gemtuzumab ozogamicin, ipilumumab, and an immune checkpoint inhibitor targeting PD-1/PD-L1.

Embodiment 53. The method of Embodiment 52, wherein the immune checkpoint inhibitor targeting PD-1/PD-L1 is chosen from nivolumab, pembrolizumab, atezolizumab, avelumab, and durvalumab.

Embodiment 54. The method of Embodiment 46, wherein the anti-cancer therapy is a targeted therapy.

Embodiment 55. The method of Embodiment 54, wherein the targeted therapy is chosen from a FLT3 inhibitor, a BCR-ABL tyrosine kinase inhibitor, a JAK-2 inhibitor, an IDH1 or IDH2 inhibitor, a BCL-2 inhibitor, and gemtuzumab ozogamicin.

Embodiment 56. The method of Embodiment 1, wherein the disease is a leukemia; wherein the cancer therapy is a chemotherapy; and wherein all of zinc, selenium, magnesium, and vitamin C are administered to the subject.

Embodiment 57. The method of Embodiment 56, wherein the leukemia is AML.

Embodiment 58. The method of Embodiment 57, wherein the chemotherapy comprises cladribine, idarubicin, and cytarabine (“CLIA”).

Embodiment 59. The method of Embodiment 58, wherein the chemotherapy comprises mylotarg, cladribine, idarubicin, and cytarabine (“CLIA-M”).

Embodiment 60. The method of any of Embodiments 47-59, wherein the method further comprises administering amifostine to the subject.

Embodiment 61. The method of any of Embodiments, 1-60, wherein the subject is a human.

Embodiment 62. The method of any of Embodiment 61, wherein the human subject:
a) has a cancer; and
b) has elevated levels of one or more metals as compared to healthy subjects.

Embodiment 63. The method of Embodiment 62, wherein elevated levels of one or more metals are measured in the bone marrow or the serum.

Embodiment 64. The method of Embodiment 62, wherein elevated levels of one or more metals are measured in the bone marrow and the serum.

Embodiment 65. The method of Embodiment 62, wherein the human subject has elevated levels of two or more of the metals.

Embodiment 66. The method of any of Embodiments 62-65, wherein the metal(s) is/are chosen from chosen from arsenic (As), aluminum (Al), antimony (Sb), Barium (Ba), boron (B), cadmium (Cd), Cerium (Ce), Chromium (Cr), lead (Pb), mercury (Hg), neodymium (Nd), Nickel (Ni), tin (Sn), titanium (Ti), uranium (U), and vanadium (V).

Embodiment 67. The method of Embodiment 62, wherein the human subject has decreased levels of at least one of calcium (Ca), magnesium (Mg), selenium (Se), zinc (Zn) and rubidium (Rb).

Embodiment 68. The method of any of Embodiments 64-66 wherein the elevated and/or reduced levels are with respect to the median values in a non-diseased population.

Embodiment 69. The method of any one of Embodiments 1-68, wherein the method results in the reduction or clearance of one or more mutations or cytogenetic abnormalities in the cancer.

Embodiment 70. A method of reduction or clearance of one or more mutations or cytogenetic abnormalities in the cancer cells of a subject with cancer, comprising administering to the subject:
(i) one or more metal chelators; and
(ii) at least one antioxidant, vitamin, or essential mineral,
in a therapeutically effective amount.

Embodiment 71. The method of Embodiment 70, wherein the method additionally comprises administering to the subject an anti-cancer therapy.

Embodiment 72. A method of reduction or clearance of one or more mutations or cytogenetic abnormalities in the cancer cells of a subject with cancer, comprising administering to the subject:
(i) one or more metal chelators; and
(ii) at least one anti-cancer therapy,
in a therapeutically effective amount.

Embodiment 73. The method of Embodiment 72, wherein the method additionally comprises administering to the subject at least one antioxidant, vitamin, and/or essential mineral.

Embodiment 74. The method of any of Embodiments 70-73, wherein the one or more metal chelators are broad-spectrum metal chelators; for example wherein at least one of the one or more metal chelators is/are capable of chelating at least two metals, and/or wherein the one or more metal chelators is/are capable of chelating potentially toxic/non-essential metal(s).

Embodiment 75. The method of Embodiment 5, wherein the one or more metal chelators is/are administered in an amount effective to reduce the levels of the at least two metals and/or in an amount effective to reduce the levels of potentially toxic/non-essential metal(s).

Embodiment 76. The method of Embodiment 6, wherein at least two chelators are administered, and the chelators are administered concurrently or sequentially.

Embodiment 77. The method of any of Embodiments 70-76, wherein the one or more metal chelators are chosen from EDTA, dimercaptosuccinic acid (DMSA), 2,3-dimercapto-1-propanesulfonic acid (DMPS), BAL, N-acetylcysteine (NAC), deferasirox, deferiprone, deferoxamine, pentetate calcium trisodium (Ca-DPTA), trientine, tetrathiomolybdate, and dexrazoxane.

Embodiment 78. The method of any of Embodiments 70-76, wherein the metal chelator is chosen from dimercaptosuccinic acid (DMSA), 2,3-dimercapto-1-propanesulfonic acid (DMPS), N-(2,3-dimercaptopropyl)-phthalamidic acid (DMPA), pentetate calcium trisodium (Ca-DTPA), and dimercaprol (BAL).

Embodiment 79. The method of any of Embodiments 70-76, wherein the metal chelator is a dithiol chelator.

Embodiment 80. The method of Embodiment 79, wherein the metal chelator is chosen from dimercaptosuccinic acid (DMSA), 2,3-dimercapto-1-propanesulfonic acid (DMPS), N-(2,3-dimercaptopropyl)-phthalamidic acid (DMPA).

Embodiment 81. The method of any of Embodiments 70-76, wherein the metal chelator is an iron chelator.

Embodiment 82. The method of Embodiment 81, wherein the metal chelator is deferasirox, deferiprone, or deferoxamine.

Embodiment 83. The method of any of Embodiments 70-76, wherein the metal chelator is chosen from N-acetyl-cysteine (NAC) and a gadolinium chelator, such as a bifunctional gadolinium(III) chelator.

Embodiment 84. The method of any of Embodiments 70-76, wherein the metal chelator is a copper chelator.

Embodiment 85. The method of Embodiment 84, wherein the copper chelator is trientine or tetrathiomolybdate.

Embodiment 86. The method of any of Embodiments 70-76, wherein the metal chelator is dexrazoxane.

Embodiment 87. The method of any of Embodiments 70-86, wherein the metal(s) chelated is/are chosen from arsenic (As), aluminum (Al), antimony (Sb), Barium (Ba), boron (B), cadmium (Cd), Cerium (Ce), Chromium (Cr), lead (Pb), mercury (Hg), neodymium (Nd), Nickel (Ni), selenium (Se), tin (Sn), titanium (Ti), uranium (U), and vanadium (V).

Embodiment 88. The method of any of Embodiments 70-86, wherein the metal(s) chelated is/are chosen from arsenic (As), antimony (Sb), boron (B), cadmium (Cd), lead (Pb), mercury (Hg), neodymium (Nd), tin (Sn), titanium (Ti), uranium (U), vanadium (V)

Embodiment 89. The method of any of Embodiments 70-86, wherein the metal(s) chelated is/are chosen from cadmium (Cd), lead (Pb), antimony (Sb), and arsenic (As).

Embodiment 90. The method of any of Embodiments 70-86, wherein the metal(s) chelated is/are chosen from antimony (Sb), cadmium (Cd), mercury (Hg), arsenic (As), and uranium (U).

Embodiment 91. The method of any of Embodiments 70-86, wherein the metal(s) chelated is/are chosen from mercury (Hg), antimony (Sb), and lead (Pb).

Embodiment 92. The method of any of Embodiments 87-91, wherein two or more metals are chelated.

Embodiment 93. The method of any of Embodiments 70-92, wherein the antioxidant(s), vitamin(s) or essential mineral(s) is/are chosen from zinc, selenium, magnesium, rubidium, and vitamin C; or the method of any of Embodiments 70-92, wherein the antioxidant(s), vitamin(s) or essential mineral(s) is/are chosen from zinc, selenium, magnesium, rubidium, ascorbic acid (vitamin C), a-tocopherol (vitamin E), glutathione, lipoic acid, uric acid, carotenoids (e.g., beta-carotene, lycopene), flavonoids (e.g. quercetin), retinol, ubiquinol (coenzyme Q), taurine, N-acetylcysteine (NAC), and amifostine.

Embodiment 94. The method of Embodiment 93, wherein at least one of zinc, selenium, magnesium, rubidium, and vitamin C are administered.

Embodiment 95. The method of Embodiment 93, wherein at least two of zinc, selenium, magnesium, rubidium, and vitamin C are administered.

Embodiment 96. The method of Embodiment 93, wherein at least three of zinc, selenium, magnesium, rubidium, and vitamin C are administered.

Embodiment 97. The method of Embodiment 93, wherein at least three of zinc, selenium, magnesium, and vitamin C are administered.

Embodiment 98. The method of Embodiment 93, wherein zinc, selenium, magnesium, rubidium, rubidium, and vitamin C are administered.

Embodiment 99. The method of Embodiment 93, wherein zinc, selenium, magnesium, and vitamin C are administered.

Embodiment 100. The method of any of Embodiments 70-99, wherein the disease is a proliferative disease.

Embodiment 101. The method of Embodiment 100, wherein the disease is chosen from cancer, a myeloproliferative neoplasm (MPN), myelodysplastic syndrome (MDS), bone marrow disease; bone marrow failure; and a cytopenia.

Embodiment 102. The method of Embodiment 101, wherein the disease is cancer.

Embodiment 103. The method of Embodiment 102, wherein the cancer has relapsed or is refractory to a previous treatment.

Embodiment 104. The method of Embodiment 103 wherein the cancer is a hematologic malignancy.

Embodiment 105. The method of Embodiment 104, wherein the cancer is a leukemia.

Embodiment 106. The method of Embodiment 105, wherein the leukemia is chosen from acute myeloid leukemia (AML), acute lymphoblastic leukemia (ALL), chronic lymphocytic leukemia (CLL), chronic myeloid leukemia (CML).

Embodiment 107. The method of Embodiment 106, wherein the leukemia is AML.

Embodiment 108. The method of Embodiment 100, wherein the disease is myelodysplastic syndrome (MDS).

Embodiment 109. The method of Embodiment 100, wherein the disease is a myeloproliferative neoplasm (MPN).

Embodiment 110. The method of Embodiment 100, wherein the disease is a bone marrow disease or bone marrow failure.

Embodiment 111. The method of Embodiment 100, wherein the disease is a cytopenia.

Embodiment 112. The method of Embodiment 111, wherein the cytopenia is an idiopathic cytopenia.

Embodiment 113. The method of any of Embodiments 70, 71, and 74-112, wherein the method additionally comprises administering to the subject a therapeutically effective amount of an anti-cancer therapy.

Embodiment 114. The method of any of Embodiments 70-113, wherein the anti-cancer therapy is chosen from a chemotherapy, an epigenetic therapy, an immunotherapy, or a targeted cancer therapy.

Embodiment 115. The method of Embodiment 113, wherein the anti-cancer therapy is an anti-cancer pharmacologic therapy.

Embodiment 116. The method of Embodiment 115, wherein the anti-cancer pharmacologic therapy comprises one or more agents chosen from mylotarg, cladribine, idarubicin, and cytarabine.

Embodiment 117. The method of Embodiment 115, wherein the anti-cancer pharmacologic therapy comprises mylotarg, cladribine, idarubicin, and cytarabine (“CLIA-M”).

Embodiment 118. The method of Embodiment 114 wherein the anti-cancer therapy is an immunotherapy.

Embodiment 119. The method of Embodiment 118, wherein the immunotherapy is chosen from a monoclonal antibody and an immune checkpoint inhibitor.

Embodiment 120. The method of Embodiment 118, wherein the immunotherapy is chosen from rituximab, ofatumumab, or blinatumomab, inotuzumab, gemtuzumab ozogamicin, ipilumumab, and an immune checkpoint inhibitor targeting PD-1/PD-L1.

Embodiment 121. The method of Embodiment 120, wherein the immune checkpoint inhibitor targeting PD-1/PD-L1 is chosen from nivolumab, pembrolizumab, atezolizumab, avelumab, and durvalumab.

Embodiment 122. The method of Embodiment 114, wherein the anti-cancer therapy is a targeted therapy.

Embodiment 123. The method of Embodiment 122, wherein the targeted therapy is chosen from a FLT3 inhibitor, a BCR-ABL tyrosine kinase inhibitor, a JAK-2 inhibitor, an IDH1 or IDH2 inhibitor, a BCL-2 inhibitor, and gemtuzumab ozogamicin.

Embodiment 124. The method of Embodiment 70, wherein the disease is a leukemia; wherein the cancer therapy is a chemotherapy; and wherein all of zinc, selenium, magnesium, and vitamin C are administered to the subject.

Embodiment 125. The method of Embodiment 124, wherein the leukemia is AML.

Embodiment 126. The method of Embodiment 125, wherein the chemotherapy comprises cladribine, idarubicin, and cytarabine (“CLIA”).

Embodiment 127. The method of Embodiment 126, wherein the chemotherapy comprises mylotarg, cladribine, idarubicin, and cytarabine (“CLIA-M”).

Embodiment 128. The method of any of Embodiments 114-127, wherein the method further comprises administering amifostine to the subject.

Embodiment 129. The method of any of Embodiments, 70-128, wherein the subject is a human.

Embodiment 130. The method of any of Embodiment 129, wherein the human subject:
a) has a cancer; and
b) has elevated levels of one or more metals as compared to healthy subjects.

Embodiment 131. The method of Embodiment 130, wherein elevated levels of one or more metals are measured in the bone marrow or the serum.

Embodiment 132. The method of Embodiment 130, wherein elevated levels of one or more metals are measured in the bone marrow and the serum.

Embodiment 133. The method of Embodiment 130, wherein the human subject has elevated levels of two or more of the metals.

Embodiment 134. The method of any of Embodiments 130-65, wherein the metal(s) is/are chosen from chosen from arsenic (As), aluminum (Al), antimony (Sb), Barium (Ba), boron (B), cadmium (Cd), Cerium (Ce), Chromium (Cr), lead (Pb), mercury (Hg), neodymium (Nd), Nickel (Ni), tin (Sn), titanium (Ti), uranium (U), and vanadium (V).

Embodiment 135. The method of Embodiment 62, wherein the human subject has decreased levels of at least one of calcium (Ca), magnesium (Mg), selenium (Se), zinc (Zn) and rubidium (Rb).

Embodiment 136. The method of any of Embodiments 64-66 wherein the elevated and/or reduced levels are with respect to the median values in a non-diseased population.

Embodiment 137. A method of diagnosing a subject with a chelation therapy -responsive proliferative disease, comprising:
i) measuring the levels of two or more metals in one or more samples of the subject's serum or bone marrow;
ii) comparing the levels of each of the two or more metals in the sample(s) to each of two or more corresponding median reference values of the same metals obtained from healthy patients; and
iii) if the levels of the two or more metals in the sample(s) are higher than the median of the reference values, classifying the subject as having a chelation therapy-responsive proliferative disease.

Embodiment 138. A method of diagnosing and treating a subject with a chelation therapy-responsive proliferative disease, comprising:
i) measuring the levels of two or more metals in one or more samples of the subject's serum or bone marrow;
ii) comparing the levels of each of the two or more metals in the sample(s) to each of two or more corresponding median reference values of the same metals obtained from healthy patients;
iii) if the levels of the two or more metals in the sample(s) higher than the median of the reference values, classifying the subject as having a chelation therapy-responsive proliferative disease; and
iv) administering to the subject:
(a) one or more metal chelators;
(b) at least one anti-cancer therapy; and
(b) optionally, at least one antioxidant and/or essential mineral,
in a therapeutically effective amount.

Embodiment 139. The method of any of Embodiments 137-138, additionally comprising:
v) measuring the levels of one or more essential minerals in one or more samples of the subject's serum or bone marrow;
vi) comparing the levels of each of the one or more essential minerals in the sample(s) to each of two or more corresponding median reference values of the same essential minerals obtained from healthy patients; and
vii) if the levels of the one or more essential minerals in the sample(s) higher than the reference values, classifying the subject as having a chelation therapy-responsive proliferative disease.

Embodiment 140. The method of any of Embodiments 137-139, wherein the metal(s) is/are chosen from arsenic (As), aluminum (Al), antimony (Sb), Barium (Ba), boron (B), cadmium (Cd), Cerium (Ce), Chromium (Cr), lead (Pb), mercury (Hg), neodymium (Nd), Nickel (Ni), selenium (Se), tin (Sn), titanium (Ti), uranium (U), and vanadium (V).

Embodiment 141. The method of any of Embodiments 137-139, wherein the metal(s) is/are chosen from arsenic (As), antimony (Sb), boron (B), cadmium (Cd), lead (Pb), mercury (Hg), neodymium (Nd), tin (Sn), titanium (Ti), uranium (U), vanadium (V)

Embodiment 142. The method of any of Embodiments 137-139, wherein the metal(s) chelated is/are chosen from cadmium (Cd), lead (Pb), antimony (Sb), and arsenic (As).

Embodiment 143. The method of any of Embodiments 137-139, wherein the metal(s) is/are chosen from antimony (Sb), cadmium (Cd), mercury (Hg), arsenic (As), and uranium (U).

Embodiment 144. The method of any of Embodiments 137-139, wherein the metal(s) is/are chosen from mercury (Hg), antimony (Sb), and lead (Pb).

Embodiment 145. The method of any of Embodiments 137-144, wherein the essential mineral(s) measured and compared is/are chosen from calcium (Ca), magnesium (Mg), selenium (Se), zinc (Zn) and rubidium (Rb).

Embodiment 146. The method of any of Embodiments 137-145, wherein three, four five, six, seven, or more metals are measured in one or more samples of the subject's serum or bone marrow.

Embodiment 147. The method of Embodiment 146, wherein elevated levels of one or more metals are measured in the bone marrow and the serum.

Embodiment 148. The method of any of Embodiments 138-147, wherein the method additionally comprises administering to the subject at least one antioxidant, vitamin, and/or essential mineral.

Embodiment 149. The method of any of Embodiments 138-148, wherein the one or more metal chelators are broad-spectrum metal chelators; for example wherein at least one of the one or more metal chelators is/are capable of chelating at least two metals, and/or wherein the one or more metal chelators is/are capable of chelating potentially toxic/non-essential metal(s).

Embodiment 150. The method of Embodiment 149, wherein the one or more metal chelators is/are administered in an amount effective to reduce the levels of the at least two metals and/or in an amount effective to reduce the levels of potentially toxic/non-essential metal(s).

Embodiment 151. The method of Embodiment 150, wherein at least two chelators are administered, and the chelators are administered concurrently or sequentially.

Embodiment 152. The method of any of Embodiments 138-151, wherein the one or more metal chelators are chosen from EDTA, dimercaptosuccinic acid (DMSA), 2,3-dimercapto-1-propanesulfonic acid (DMPS), BAL, N-acetylcysteine (NAC), deferasirox, deferiprone, deferoxamine, pentetate calcium trisodium (Ca-DPTA), trientine, tetrathiomolybdate, and dexrazoxane.

Embodiment 152. The method of any of Embodiments 138-151, wherein the metal chelator is chosen from dimercaptosuccinic acid (DMSA), 2,3-dimercapto-1-propanesulfonic acid (DMPS), N-(2,3-dimercaptopropyl)-phthalamidic acid (DMPA), pentetate calcium trisodium (Ca-DTPA), and dimercaprol (BAL).

Embodiment 153. The method of any of Embodiments 138-151, wherein the metal chelator is a dithiol chelator.

Embodiment 154. The method of Embodiment 153, wherein the metal chelator is chosen from dimercaptosuccinic acid (DMSA), 2,3-dimercapto-1-propanesulfonic acid (DMPS), N-(2,3-dimercaptopropyl)-phthalamidic acid (DMPA).

Embodiment 155. The method of any of Embodiments 138-151, wherein the metal chelator is an iron chelator.

Embodiment 156. The method of Embodiment 155, wherein the metal chelator is deferasirox, deferiprone, or deferoxamine

Embodiment 157. The method of any of Embodiments 138-151, wherein the metal chelator is chosen from N-acetyl-cysteine (NAC) and a gadolinium chelator, such as a bifunctional gadolinium(III) chelator.

Embodiment 158. The method of any of Embodiments 138-151, wherein the metal chelator is a copper chelator.

Embodiment 159. The method of Embodiment 16, wherein the copper chelator is trientine or tetrathiomolybdate.

Embodiment 160. The method of any of Embodiments 138-151, wherein the metal chelator is dexrazoxane.

Embodiment 161. The method of any of Embodiments 138-160, wherein the metal(s) chelated is/are chosen from arsenic (As), aluminum (Al), antimony (Sb), Barium (Ba), boron (B), cadmium (Cd), Cerium (Ce), Chromium (Cr), lead (Pb), mercury (Hg), neodymium (Nd), Nickel (Ni), selenium (Se), tin (Sn), titanium (Ti), uranium (U), and vanadium (V).

Embodiment 162. The method of any of Embodiments 138-160, wherein the metal(s) chelated is/are chosen from arsenic (As), antimony (Sb), boron (B), cadmium (Cd), lead (Pb), mercury (Hg), neodymium (Nd), tin (Sn), titanium (Ti), uranium (U), vanadium (V)

Embodiment 163. The method of any of Embodiments 138-160, wherein the metal(s) chelated is/are chosen from cadmium (Cd), lead (Pb), antimony (Sb), and arsenic (As).

Embodiment 164. The method of any of Embodiments 138-160, wherein the metal(s) chelated is/are chosen from antimony (Sb), cadmium (Cd), mercury (Hg), arsenic (As), and uranium (U).

Embodiment 165. The method of any of Embodiments 138-160, wherein the metal(s) chelated is/are chosen from mercury (Hg), antimony (Sb), and lead (Pb).

Embodiment 166. The method of any of Embodiments 138-165, wherein two or more metals are chelated.

Embodiment 167. The method of any of Embodiments 138-166, wherein the antioxidant(s), vitamin(s), or essential mineral(s) administered is/are chosen from zinc, selenium, magnesium, rubidium, and vitamin C; or the method of any of Embodiments 138-166, wherein the antioxidant(s), vitamin(s) or essential mineral(s) is/are chosen from zinc, selenium, magnesium, rubidium, ascorbic acid (vitamin C), α-tocopherol (vitamin E), glutathione, lipoic acid, uric acid, carotenoids (e.g., beta-carotene, lycopene), flavonoids (e.g. quercetin), retinol, ubiquinol (coenzyme Q), taurine, N-acetylcysteine (NAC), and amifostine.

Embodiment 168. The method of Embodiment 167, wherein at least one of zinc, selenium, magnesium, rubidium, and vitamin C are administered.

Embodiment 169. The method of Embodiment 167, wherein at least two of zinc, selenium, magnesium, rubidium, and vitamin C are administered.

Embodiment 170. The method of Embodiment 167, wherein at least three of zinc, selenium, magnesium, rubidium, and vitamin C are administered.

Embodiment 171. The method of Embodiment 167, wherein at least three of zinc, selenium, magnesium, and vitamin C are administered.

Embodiment 172. The method of Embodiment 167, wherein zinc, selenium, magnesium, rubidium, rubidium, and vitamin C are administered.

Embodiment 173. The method of Embodiment 167, wherein zinc, selenium, magnesium, and vitamin C are administered.

Embodiment 174. The method of any of Embodiments 138-173, wherein the disease is a proliferative disease.

Embodiment 175. The method of Embodiment 174, wherein the disease is chosen from cancer, a myeloproliferative neoplasm (MPN), myelodysplastic syndrome (MDS), bone marrow disease; bone marrow failure; and a cytopenia.

Embodiment 176. The method of Embodiment 175, wherein the disease is cancer.

Embodiment 177. The method of Embodiment 176, wherein the cancer has relapsed or is refractory to a previous treatment.

Embodiment 178. The method of Embodiment 177 wherein the cancer is a hematologic malignancy.

Embodiment 179. The method of Embodiment 178, wherein the cancer is a leukemia.

Embodiment 180. The method of Embodiment 179, wherein the leukemia is chosen from acute myeloid leukemia (AML), acute lymphoblastic leukemia (ALL), chronic lymphocytic leukemia (CLL), chronic myeloid leukemia (CML).

Embodiment 181. The method of Embodiment 180, wherein the leukemia is AML.

Embodiment 182. The method of Embodiment 175, wherein the disease is myelodysplastic syndrome (MDS).

Embodiment 183. The method of Embodiment 175, wherein the disease is a myeloproliferative neoplasm (MPN).

Embodiment 184. The method of Embodiment 175, wherein the disease is a bone marrow disease or bone marrow failure.

Embodiment 185. The method of Embodiment 175, wherein the disease is a cytopenia.

Embodiment 186. The method of Embodiment 185, wherein the cytopenia is an idiopathic cytopenia.

Embodiment 187. The method of any of Embodiments 138-186, wherein the anti-cancer therapy is chosen from a chemotherapy, an epigenetic therapy, an immunotherapy, or a targeted cancer therapy.

Embodiment 188. The method of Embodiment 187, wherein the anti-cancer therapy is an anti-cancer pharmacologic therapy.

Embodiment 189. The method of Embodiment 188, wherein the anti-cancer pharmacologic therapy comprises one or more agents chosen from mylotarg, cladribine, idarubicin, and cytarabine.

Embodiment 190. The method of Embodiment 188, wherein the anti-cancer pharmacologic therapy comprises mylotarg, cladribine, idarubicin, and cytarabine (“CLIA-M”).

Embodiment 191. The method of Embodiment 187 wherein the anti-cancer therapy is an immunotherapy.

Embodiment 192. The method of Embodiment 191, wherein the immunotherapy is chosen from a monoclonal antibody and an immune checkpoint inhibitor.

Embodiment 193. The method of Embodiment 191, wherein the immunotherapy is chosen from rituximab, ofatumumab, or blinatumomab, inotuzumab, gemtuzumab ozogamicin, ipilumumab, and an immune checkpoint inhibitor targeting PD-1/PD-L1.

Embodiment 194. The method of Embodiment 193, wherein the immune checkpoint inhibitor targeting PD-1/PD-L1 is chosen from nivolumab, pembrolizumab, atezolizumab, avelumab, and durvalumab.

Embodiment 195. The method of Embodiment 187, wherein the anti-cancer therapy is a targeted therapy.

Embodiment 196. The method of Embodiment 195, wherein the targeted therapy is chosen from a FLT3 inhibitor, a BCR-ABL tyrosine kinase inhibitor, a JAK-2 inhibitor, an IDH1 or IDH2 inhibitor, a BCL-2 inhibitor, and gemtuzumab ozogamicin.

Embodiment 197. The method of Embodiment 138, wherein the disease is a leukemia; wherein the cancer therapy is a chemotherapy; and wherein all of zinc, selenium, magnesium, and vitamin C are administered to the subject.

Embodiment 198. The method of Embodiment 197, wherein the leukemia is AML.

Embodiment 199. The method of Embodiment 198, wherein the chemotherapy comprises cladribine, idarubicin, and cytarabine (“CLIA”).

Embodiment 200. The method of Embodiment 199, wherein the chemotherapy comprises mylotarg, cladribine, idarubicin, and cytarabine (“CLIA-M”).

Embodiment 201. The method of any of Embodiments 197-200, wherein the method further comprises administering amifostine to the subject.

Embodiment 202. The method of any of Embodiments, 138-201, wherein the subject is a human.

Embodiment 203. The method of any one of Embodiments 138-202, wherein the method results in the reduction or clearance of one or more mutations or cytogenetic abnormalities in the cancer.

Embodiment 204. A pharmaceutical composition or a pharmaceutical combination, comprising
(i) one or more metal chelators;
(ii) at least one antioxidant, vitamin, or essential mineral; and
(iii) a pharmaceutically acceptable excipient.

Embodiment 205. The pharmaceutical composition or the pharmaceutical combination of Embodiment 204 additionally comprising at least one anti-cancer pharmacologic therapy.

Embodiment 206. A pharmaceutical composition or a pharmaceutical combination, comprising
(i) one or more metal chelators;
(ii) at least one anti-cancer pharmacologic therapy; and
(iii) a pharmaceutically acceptable excipient.

Embodiment 207. The pharmaceutical composition or the pharmaceutical combination of Embodiment 206 additionally comprising at least one antioxidant, vitamin, or essential mineral.

Embodiment 208. A metal chelator for use in a method of treating cancer in a mammalian subject, wherein said method comprises administering a cancer therapy to said subject.

Embodiment 209. A cancer therapy for use in a method of treating cancer in a mammalian subject, wherein said method comprises administering a metal chelator to said subject.

Embodiment 210. An antioxidant and/or mineral, preferably an essential mineral, selected from zinc, selenium, magnesium, rubidium and vitamin C, for use in a method of treating cancer, wherein said method comprises administering a metal chelator and/or a cancer therapy.

Embodiment 211. A metal chelator for use in a method of treating cancer in a mammalian subject, wherein said metal chelator is for administration in combination with a cancer therapy.

Embodiment 212. A cancer therapy for use in a method of treating cancer in a mammalian subject, wherein said cancer therapy is for administration in combination with a metal chelator.

Embodiment 213. An antioxidant and/or mineral, preferably an essential mineral, selected from zinc, selenium, magnesium, rubidium and vitamin C, for use in a method of treating cancer, wherein said antioxidant and/or mineral is for administration in combination with a metal chelator and/or a cancer therapy.

Embodiment 214. The pharmaceutical composition, the pharmaceutical combination, the metal chelator for use, the cancer therapy for use, or the antioxidant and/or mineral for use of any of Embodiments 204-213, wherein the antioxidant(s), vitamin(s), or mineral(s) is/are chosen from zinc, selenium, magnesium, rubidium, and vitamin C; or the method of any of Embodiments 204-213, wherein the antioxidant(s), vitamin(s) or essential mineral(s) is/are chosen from zinc, selenium, magnesium, rubidium, ascorbic acid (vitamin C), a-tocopherol (vitamin E), glutathione, lipoic acid, uric acid, carotenoids (e.g., beta-carotene, lycopene), flavonoids (e.g. quercetin), retinol, ubiquinol (coenzyme Q), taurine, N-acetylcysteine (NAC), and amifostine.

Embodiment 215. The pharmaceutical composition, the pharmaceutical combination the metal chelator for use, the cancer therapy for use, or the antioxidant and/or mineral for use of any of Embodiments 204-214, wherein the anti-cancer therapy is chosen from a chemotherapy, an epigenetic therapy, an immunotherapy, or a targeted cancer therapy.

Embodiment 216. The pharmaceutical composition, the pharmaceutical combination, the metal chelator for use, the cancer therapy for use, or the antioxidant and/or mineral for use of Embodiment 215, wherein the anti-cancer therapy is an anti-cancer pharmacologic therapy.

Embodiment 217. The pharmaceutical composition, the pharmaceutical combination, the metal chelator for use, the cancer therapy for use, or the antioxidant and/or mineral for use of Embodiment 216, wherein the anti-cancer pharmacologic therapy comprises one or more agents chosen from mylotarg, cladribine, idarubicin, and cytarabine.

Embodiment 218. The pharmaceutical composition, the pharmaceutical combination, the metal chelator for use, the cancer therapy for use, or the antioxidant and/or mineral for use of Embodiment 216, wherein the anti-cancer pharmacologic therapy comprises mylotarg, cladribine, idarubicin, and cytarabine (“CLIA-M”) or cladribine, idarubicin, and cytarabine (“CLIA”).

Embodiment 219. The pharmaceutical composition, the pharmaceutical combination, the metal chelator for use, the cancer therapy for use, or the antioxidant and/or mineral for use of Embodiment 215 wherein the anti-cancer therapy is an immunotherapy.

Embodiment 220. The pharmaceutical composition, the pharmaceutical combination, the metal chelator for use, the cancer therapy for use, or the antioxidant and/or mineral for use of Embodiment 219, wherein the immunotherapy is chosen from a monoclonal antibody and an immune checkpoint inhibitor.

Embodiment 221. The pharmaceutical composition, the pharmaceutical combination, the metal chelator for use, the cancer therapy for use, or the antioxidant and/or mineral for use of Embodiment 219, wherein the immunotherapy is chosen from rituximab, ofatumumab, or blinatumomab, inotuzumab, gemtuzumab ozogamicin, ipilumumab, and an immune checkpoint inhibitor targeting PD-1/PD-L1.

Embodiment 222. The pharmaceutical composition, the pharmaceutical combination, the metal chelator for use, the cancer therapy for use, or the antioxidant and/or mineral for use of Embodiment 220, wherein the immune checkpoint inhibitor targeting PD-1/PD-L1 is chosen from nivolumab, pembrolizumab, atezolizumab, avelumab, and durvalumab.

Embodiment 223. The pharmaceutical composition, the pharmaceutical combination, the metal chelator for use, the cancer therapy for use, or the antioxidant and/or mineral for use of Embodiment 215, wherein the anti-cancer therapy is a targeted therapy.

Embodiment 224. The pharmaceutical composition, the pharmaceutical combination, the metal chelator for use, the cancer therapy for use, or the antioxidant and/or mineral for use of Embodiment 223, wherein the targeted therapy is chosen from a FLT3 inhibitor, a BCR-ABL tyrosine kinase inhibitor, a JAK-2 inhibitor, an IDH1 or IDH2 inhibitor, a BCL-2 inhibitor, and gemtuzumab ozogamicin.

Embodiment 225. The pharmaceutical composition, the pharmaceutical combination, the metal chelator for use, the cancer therapy for use, or the antioxidant and/or mineral for use of any of Embodiments 204-224, wherein the one or more metal chelators are broad-spectrum metal chelators; for example wherein at least one of the one or more metal chelators is/are capable of chelating at least two metals, and/or wherein the one or more metal chelators is/are capable of chelating potentially toxic/non-essential metal(s).

Embodiment 226. The pharmaceutical composition, the pharmaceutical combination, the metal chelator for use, the cancer therapy for use, or the antioxidant and/or mineral for use of Embodiment 225, wherein the one or more metal chelators is/are administered in an amount effective to reduce the levels of the at least two metals and/or in an amount effective to reduce the levels of potentially toxic/non-essential metal(s).

Embodiment 227. The pharmaceutical composition, the pharmaceutical combination, the metal chelator for use, the cancer therapy for use, or the antioxidant and/or mineral for use of Embodiment 226, wherein at least two chelators are administered, and the chelators are administered concurrently or sequentially.

Embodiment 228. The pharmaceutical composition, the pharmaceutical combination, the metal chelator for use, the cancer therapy for use, or the antioxidant and/or mineral for use of any of Embodiments 204-227, wherein the one or more metal chelators are chosen from EDTA, dimercaptosuccinic acid (DMSA), 2,3-dimercapto-1-propanesulfonic acid (DMPS), BAL, N-acetylcysteine (NAC), deferasirox, deferiprone, deferoxamine, pentetate calcium trisodium (Ca-DPTA), trientine, tetrathiomolybdate, and dexrazoxane.

Embodiment 229. The pharmaceutical composition, the pharmaceutical combination, the metal chelator for use, the cancer therapy for use, or the antioxidant and/or mineral for use of any of Embodiments 204-227, wherein the metal chelator is chosen from dimercaptosuccinic acid (DMSA), 2,3-dimercapto-1-propanesulfonic acid (DMPS), N-(2,3-dimercaptopropyl)-phthalamidic acid (DMPA), pentetate calcium trisodium (Ca-DTPA), and dimercaprol (BAL).

Embodiment 230. The pharmaceutical composition, the pharmaceutical combination, the metal chelator for use, the cancer therapy for use, or the antioxidant and/or mineral for use of any of Embodiments 204-227, wherein the metal chelator is a dithiol chelator.

Embodiment 231. The pharmaceutical composition, the pharmaceutical combination, the metal chelator for use, the cancer therapy for use, or the antioxidant and/or mineral for use of Embodiment 230, wherein the metal chelator is chosen from dimercaptosuccinic acid (DMSA), 2,3-dimercapto-1-propanesulfonic acid (DMPS), N-(2,3-dimercaptopropyl)-phthalamidic acid (DMPA).

Embodiment 232. The pharmaceutical composition, the pharmaceutical combination, the metal chelator for use, the cancer therapy for use, or the antioxidant and/or mineral for use of any of Embodiments 204-227, wherein the metal chelator is an iron chelator.

Embodiment 233. The pharmaceutical composition, the pharmaceutical combination, the metal chelator for use, the cancer therapy for use, or the antioxidant and/or mineral for use of Embodiment 232, wherein the metal chelator is deferasirox, deferiprone, or deferoxamine.

Embodiment 234. The pharmaceutical composition, the pharmaceutical combination, the metal chelator for use, the cancer therapy for use, or the antioxidant and/or mineral for use of any of Embodiments 204-227, wherein the metal chelator is chosen from N-acetyl-cysteine (NAC) and a gadolinium chelator, such as a bifunctional gadolinium(III) chelator.

Embodiment 235. The pharmaceutical composition, the pharmaceutical combination, the metal chelator for use, the cancer therapy for use, or the antioxidant and/or mineral for use of any of Embodiments 204-227, wherein the metal chelator is a copper chelator.

Embodiment 236. The pharmaceutical composition, the pharmaceutical combination, the metal chelator for use, the cancer therapy for use, or the antioxidant and/or mineral for use of Embodiment 235, wherein the copper chelator is trientine or tetrathiomolybdate.

Embodiment 237. The pharmaceutical composition, the pharmaceutical combination, the metal chelator for use, the cancer therapy for use, or the antioxidant and/or mineral for use of any of Embodiments 204-227, wherein the metal chelator is dexrazoxane.

Embodiment 238. The pharmaceutical composition, the pharmaceutical combination of any one of Embodiments 204-207 and 214-237, wherein the pharmaceutical composition or pharmaceutical combination is formulated for oral, subcutaneous, or intravenous administration.

Embodiment 239. The pharmaceutical composition or pharmaceutical combination of Embodiment 238, wherein the pharmaceutical composition or combination is formulated for oral administration.

Embodiment 240. The pharmaceutical composition or pharmaceutical combination of Embodiment 238, wherein the pharmaceutical composition or combination is formulated for subcutaneous administration.

Embodiment 241. The pharmaceutical composition or the pharmaceutical combination of Embodiment 238, wherein the pharmaceutical composition or pharmaceutical combination is formulated for intravenous administration.

Also provided are the following embodiments.

Embodiment P1. A method of treating a disease in a mammalian subject comprising administering to the subject therapeutically effective amounts of:
(i) a metal chelator;
(ii) a cancer therapy, wherein the cancer therapy is a chemotherapy, an epigenetic therapy, an immunotherapy, or a targeted cancer therapy; and
(iii) at least one of, more preferably at least two of, more preferably at least three of, and even more preferably all of zinc, selenium, magnesium, and/or vitamin C;
wherein the disease is preferably a cancer.

Embodiment P2. The method of Embodiment P1, wherein if the chelator is dexrazoxane, then method comprises administering a second metal chelator to the subject.

Embodiment P3. The method of Embodiment P1, wherein the cancer is a leukemia or a hematological malignancy.

Embodiment P3. The method of Embodiment P3, wherein the leukemia is acute myeloid leukemia (AML), acute lymphoblastic leukemia (ALL), chronic lymphocytic leukemia (CLL), or chronic myeloid leukemia (CML).

Embodiment P4. The method of Embodiment P4, wherein the subject has myelodysplastic syndrome (MDS).

Embodiment P5. The method of Embodiment P4, wherein the cancer has relapsed or is refractory to a previous treatment.

Embodiment P6. The method of Embodiment P1, wherein the metal chelator is a dithiol chelator, preferably dimercaptosuccinic acid (DMSA), 2,3-dimercapto-1-propanesulfonic acid (DMPS), N-(2,3-dimercaptopropyl)-phthalamidic acid (DMPA).

Embodiment P7. The method of Embodiment P1, wherein the chelator is dimercaptosuccinic acid (DMSA), 2,3-dimercapto-1-propanesulfonic acid (DMPS), N-(2,3-dimercaptopropyl)-phthalamidic acid (DMPA), pentetate calcium trisodium (Ca-DTPA), or dimercaprol (BAL).

Embodiment P8. The method of Embodiment P8, wherein the method comprises administering both dimercaptosuccinic acid (DMSA) and 2,3-dimercapto-1-propanesulfonic acid (DMPS) to the subject.

Embodiment P9. The method of Embodiment P1, wherein the metal chelator is calcium-EDTA (Ca-EDTA) or calcium disodium EDTA.

Embodiment P10. The method of Embodiment P10, wherein the metal chelator is an iron chelator.

Embodiment P11. The method of Embodiment P11, wherein the metal chelator is deferasirox, deferiprone, or deferoxamine

Embodiment P12. The method of Embodiment P1, wherein the metal chelator is N-acetyl-cysteine (NAC) or a gadolinium chelator, such as a bifunctional gadolinium(III) chelator.

Embodiment P13. The method of Embodiment P1, wherein the metal chelator is a copper chelator.

Embodiment P14. The method of Embodiment P14, wherein the copper chelator is trientine or tetrathiomolybdate.

Embodiment P15. The method of Embodiment P1, wherein the metal chelator is dexrazoxane.

Embodiment P16. The method of any one of Embodiments P1-P15, wherein the method further comprises administering to the subject a therapeutically effective dose of dexrazoxane.

Embodiment P17. The method of any one of Embodiments P1-P15, wherein the method further comprises administering to the subject a therapeutically effective dose of amifostine.

Embodiment P18. The method of Embodiment P17, wherein the method comprises administering zinc, selenium, magnesium, and vitamin C to the subject.

Embodiment P19. The method of any one of Embodiments P1-P16, wherein the method comprises administering zinc, selenium, magnesium, and vitamin C to the subject.

Embodiment P20. The method of any one of Embodiments P1-P16, wherein the cancer therapy is a chemotherapy.

Embodiment P21. The method of Embodiment P21, wherein the chemotherapy is mylotarg, cladribine, idarubicin, or cytarabine.

Embodiment P22. The method of Embodiment P22, wherein the chemotherapy is mylotarg, cladribine, idarubicin, and cytarabine (CLIA-M).

Embodiment P23. The method of Embodiment P21, wherein the method further comprises administering Amifostine to the subject.

Embodiment P24. The method of Embodiment P1, wherein the cancer is a leukemia, preferably AML; wherein the cancer therapy is a chemotherapy; and wherein all of zinc, selenium, magnesium, and vitamin C are administered to the subject.

Embodiment P25. The method of Embodiment P25, wherein the chemotherapy is an anthracycline, a Berlin-Frankfurt-Munster (BFM) chemotherapy, a hyper-CVAD chemotherapy comprising (cyclophosphamide, vincristine, doxorubicin, and dexamethasone), a hypomethylating therapy (e.g., decitabine or azacytidine), cytarabine, clofarabine, or cladribine.

Embodiment P26. The method of Embodiment P1 wherein the cancer therapy is an immunotherapy.

Embodiment P27. The method of Embodiment P27, wherein the immunotherapy is a monoclonal antibody or an immune checkpoint inhibitor.

Embodiment P28. The method of Embodiment P27, wherein the immunotherapy is rituximab, ofatumumab, or blinatumomab, inotuzumab, gemtuzumab ozogamicin, nivolumab, ipilumumab, or an immune checkpoint inhibitor targeting PD-1/PD-L1.

Embodiment P29. The method of Embodiment P1, wherein the cancer therapy is a targeted therapy.

Embodiment P30. The method of Embodiment P30, wherein the targeted therapy is a FLT3 inhibitor, a BCR-ABL tyrosine kinase inhibitor, a JAK-2 inhibitor, an IDH1 or IDH2 inhibitor, gemtuzumab ozogamicin.

Embodiment P31. The method of any one of Embodiments P1-P16, wherein the subject is a human.

Embodiment P32. The method of any one of Embodiments P1-P32, wherein the method results in the reduction or clearance of one or more mutations or cytogenetic abnormalities in the cancer.

Embodiment P33. A pharmaceutical composition comprising (i) a metal chelator and (ii) at least two of, more preferably at least three of, and even more preferably all of zinc, selenium, magnesium, and/or vitamin C, and a pharmaceutically acceptable excipient.

Embodiment P34. The pharmaceutical composition of Embodiment P34, wherein the metal chelator is dimercaptosuccinic acid (DMSA), 2,3-dimercapto-1-propanesulfonic acid (DMPS), dimercaprol (BAL), calcium-EDTA (Ca-EDTA), pentetate calcium trisodium (Ca-DTPA), N-(2,3-dimercaptopropyl)-phthalamidic acid (DMPA), or calcium disodium EDTA.

Embodiment P35. The pharmaceutical composition of any one of Embodiments P34-P35, wherein the pharmaceutical composition is formulated for oral or intravenous administration.

### I. COMBINATION THERAPIES AND METHODS

A. Metal Chelators

The present methods and compositions can comprise one or more metal chelating agents, such as broad-spectrum metal chelators or specific metal chelators. Metal chelators known in the art may be used including, but not limited to, calcium ethylenediaminetetraacetic acid (EDTA), dithiol chelators, iron chelators, and copper chelators. When more than one metal chelator is administered to the subject, the metal chelators may be co-administered at the same time (e.g., in a single formulation or in separate formulations) or administered sequentially (e.g., in a single formulation or in separate formulations).

The metal chelators may be administered for the treatment of diseases disclosed herein, such as proliferative diseases, including cancers, chosen from: leukemias, such as acute myeloid leukemia (AML), acute lymphoblastic leukemia (ALL), chronic lymphocytic leukemia (CLL), chronic myeloid leukemia (CML); myelodysplastic syndromes (MDSs); myeloproliferative neoplasms (MPNs); bone marrow disease; bone marrow failure; and cytopenia (including idiopathic cytopenias). The metal chelators may be administered for the treatment of a cancer, e.g., a leukemia, such as acute myeloid leukemia (AML), acute lymphoblastic leukemia (ALL), chronic lymphocytic leukemia (CLL), or chronic myeloid leukemia (CML). The therapy may be administered during leukemia induction or consolidation therapy and/or maintenance therapy. The therapy may be administered during the course of therapy of myeloproliferative neoplasms or aplastic anemia therapy.

EDTA, such as preferably calcium EDTA or calcium disodium EDTA, is a broad-spectrum chelator that can remove heavy metals and minerals from the blood. EDTA (e.g., calcium EDTA) may be administered intravenously. Ca-EDTA may be given as 1 g/m² in normal saline or at multiple other doses as described previously (Calcium disodium versenate, 2013). The dose of calcium EDTA may be about 0.5 g/m² to about 5 g/m², such as about 1 g/m², 2 g/m², 3 g/m², 4 g/m², or 5 g/m², preferably about 1 g/m². The calcium EDTA can be administered in normal saline.

The metal chelator(s) may be a dithiol chelator, such as for example dimercaptosuccinic acid (DMSA; also known as Succimer) and/or 2,3-dimercapto-1-propanesulfonic acid (DMPS). Oral DMSA administered at about 30 mg/kg/day can provide an effective antidote for lead poisoning; however, wide inter- and intra-individual variation can be used (Bradberry et al., 2009; Package Insert, 2007). DMPS-DMSA can be alternated or replaced with DMPS (Bose-O'Reilly et al., 2003) at a dose of about 200-400 mg daily, depending on the clinical context. The DMSA can be administered orally, such as at a dose of 5-50 mg/kg/day, such as about 10 mg/kg/day, 20 mg/kg/day, 30 mg/kg/day, 40 mg/kg/day, or 50 mg/kg/day, preferably about 30 mg/kg/day. DMPS can be administered orally, such as at a dose of 200-400 mg/day, such as about 200 mg/day, 250 mg/day, 300 mg/day, 350 mg/day, or 400 mg/day. Dimercaprol (also named British Anti-Lewisite or BAL), is another organic dithiol compound (Flora and Pachauri, 2010).

BAL is typically administered by deep intramuscular injection. BAL may be administered as follows: 2.5 mg/kg of body weight four times daily for two days, two times on the third day, and once daily thereafter for ten days (FDA, Dimercaprol Injection). Alternatively, BAL may be given 3 mg/kg every four hours for two-days, four times on the third day, then twice daily thereafter for ten days. Alternatively, BAL may be administered 5 mg/kg initially, followed by 2.5 mg/kg one to two times/day for ten days. BAL may also be administered as follows: 4 mg/kg body weight is given alone in the first dose and thereafter at four-hour intervals in combination with Edetate Calcium Disodium Injection USP (to be administered at a separate site). The dose can be reduced to 3 mg/kg after the first dose. In one embodiment, treatment is maintained for two to seven days depending on clinical response.

Iron chelators may be used in the present methods and compositions. Exemplary iron chelators include, but are not limited to, deferasirox (Exjade; Jadenu), deferiprone (Ferriprox), and deferoxamine (Desferal). The deferasirox or dexrazoxane may be administered at an exemplary dose of 10-50 mg/kg/day, such as about 10 mg/kg/day, 20 mg/kg/day, 30 mg/kg/day, 40 mg/kg/day, or 50 mg/kg/day, preferably about 20-40 mg/kg/day. The deferiprone may be administered (e.g., orally) at an exemplary dose of 20-100 mg/kg, such as 40-90 mg/kg. The deferoxamine may be administered at an exemplary 1 to 2 g dosing up to 5-7 days or may be infused overnight (e.g. subcutaneously). The iron chelators may be administered subcutaneously, intravenously, or intramuscularly, and for some of the chelators, orally.

Another exemplary metal chelator which may be used in the present methods and compositions is N-acetyl-cysteine (NAC). NAC may be administered intravenously or orally. An exemplary intravenous dose may be about 100-300 mg/mL, particularly 200 mg/mL. An exemplary oral dose of NAC may comprise 250-1000 mg tablets, such as 500 mg, 600 mg, or 700 mg tablets. As an example, the tablets may be administered once, twice, or three times a day; in certain embodiments, they may be administered once or twice a day. The NAC may be administered both intravenously and orally.

Gadolinium chelators may be used in the present methods. For example, in some embodiments, a bifunctional gadolinium (III) chelator may be administered to a subject (e.g., Frullano, 2011).

Pentetate calcium trisodium (Ca-DTPA) and pentetate zinc trisodium (Zn-DTPA) are chelators that may be used in the present methods. Ca-DTPA contains the sodium salt of calcium diethylenetriaminepentaacetate and is also known as trisodium calcium diethylenetriaminepentaacetate. Ca-DTPA and Zn-DPTA can be administered intravenously (slow push or infusion) or via inhalation. Generally, the same dose and dose schedule is used for Zn-DTPA as for Ca-DTPA. They may be administered for chelation therapy either as an infusion at 1 g in 100-250 cc D5W or NS over 30 minutes, or as a slow IV push at 1 g in 5 cc 5% dextrose in water (D5W) or 0.9% sodium chloride (normal saline, NS) over 3-4 minutes, or via a nebulizer at 1 g in 1:1 dilution with sterile water or NS, inhaled over 15-20 minutes.

Copper chelators may be used in the present methods including, but not limited to, trientine (Syprine), tetrathiomolybdate, and dexrazoxane. Trientine may be administered at an exemplary dose of 750-1250 mg/day given in divided doses two, three, or four times daily. As an example, trientine may be administered orally, such as 250 mg or 500 mg tablets. Dose adjustments may be made as necessary to maintain serum ceruloplasmin levels at 5-15 mg/dL. Tetrathiomolybdate may be administered at an exemplary dose of 100-200, such as 180 mg, in divided doses, such as 4 divided doses, and may be adjusted to maintain a copper level of 5-15 mg/dL. Dexrazoxane may be administered in combination with anthracyclines. Dexrazoxane (Totect; Savene) can be given at an exemplary intravenous dose of 500-1000 mg/m2 for a maximum daily dose of 1000-2000 mg.

B. Antioxidants, Minerals, and Vitamins

Many embodiments herein comprise antioxidants, vitamins, and/or essential minerals, administered in combination with anti-cancer therapy and/or chelators. Bioactive antioxidants include certain compounds also classified as vitamins Antioxidant vitamins include ascorbic acid (vitamin C) and a-tocopherol (vitamin E). Other antioxidant compounds include glutathione, lipoic acid, uric acid, carotenoids (e.g., beta-carotene, lycopene), flavonoids (e.g. quercetin), retinol, ubiquinol (coenzyme Q), taurine, N-acetylcysteine (NAC), and amifostine. Antioxidants can be generally hydrophilic or generally lipophilic; in certain embodiments one or the other will be appropriate.

Essential minerals are those minerals which are necessary for proper functioning of the body. They are sometimes classified into i) macrominerals such as sodium, chloride, potassium, calcium, phosphorus, magnesium, and sulfur; and microminerals (trace minerals) such as iron, zinc, iodine, selenium, copper, manganese, fluoride, and molybdenum. In certain embodiments, essential minerals can include zinc, selenium, magnesium, calcium, rubidium, and copper. One subset of such minerals is zinc, selenium, magnesium, calcium, and rubidium; a further subset is zinc, selenium, magnesium, and calcium.

In some embodiments, at least one, more preferably two, more preferably three, and even more preferably all of the following vitamins and minerals are administered to a subject: zinc, selenium, magnesium, and/or vitamin C; preferably, these compounds are administered to the subject in combination with an additional anticancer therapy as described herein. Examples of ranges of dosages and forms of zinc, selenium, magnesium, and vitamin C that can be administered to a subject or patient can include but are not limited to the following: zinc (e.g., elemental zinc, zinc sulfate, zinc citrate, or zinc glycenate at 25-75 mg, such as 50 mg), vitamin C (e.g., 1000 mg to 50 grams, such as orally or intravenously), magnesium citrate (e.g., 100 mg to 3 g, such as orally or intravenously, such as 3 g magnesium sulfate IV), and selenium (e.g., L-Selenomethionine or equivalent, such as 100-200 mcg orally daily).

In some embodiments, all of the following vitamins and minerals are administered to a subject zinc, selenium, magnesium, rubidium, and/or vitamin C; preferably, these compounds are administered to the subject in combination with an additional anticancer therapy as described herein.

C. Methods of Treatment

Certain embodiments of the present disclosure provide methods for treating and/or preventing cancer, such as leukemia, and other diseases with metal chelation drugs. The metal chelation drugs may be administered alone or in combination with antioxidants, mineral, vitamins, and/or free radical scavenging agents. Further combination therapies include chemotherapy, immunotherapy, and targeted therapy.

Methods of treating cancer may comprise administering metal chelation drugs, antioxidants, minerals, vitamins, free radical scavenging agents, and/or chemotherapy during the course of treatment, as maintenance therapy, or as cancer prevention. In some aspects, trace mineral supplements, such as magnesium, selenium, zinc, and vitamin C, may be administered during cancer treatment, cancer maintenance therapy, or for disease prevention.

Metal chelation therapy may be used for detoxification in a healthy subject for health maintenance. The metal chelation therapy may be combined with antioxidants, minerals, vitamins, and/or free radical scavenging agents for rebalancing.

The chelating agents disclosed herein are examples of chelating agents which may be administered to a subject alone or in combination with antioxidants, vitamins, and/or minerals as disclosed herein. They may be given concurrently or sequentially. In some embodiments, one or more of the following chelating agents. In some embodiments, one or more chelating agent(s) are preferably combined with antioxidants/mineral vitamins and given with an additional cancer therapy (e.g., chemotherapy/epigenetic therapy/immunotherapy/targeted therapy during cancer treatment, cancer maintenance therapy, or cancer prevention therapy). The chelating agent +antioxidant/mineral/vitamin combinations may comprise of one or more chelating agent and may be utilized with or without chemotherapy/epigenetic therapy/immunotherapy/targeted therapy during cancer treatment, cancer maintenance therapy, or cancer prevention. These chelators may be given at a variety of dosages including but not limited to the following ranges or schedules.

Calcium-EDTA (Ca-EDTA): can be used for broad-spectrum chelation As an example, Ca-EDTA may be given as 1 g/m2 in normal saline or at multiple other doses as described previously (Calcium disodium versenate, 2013).

DMSA and/or DMPS: As an example, oral DMSA administered at about 30 mg/kg/day can provide an effective antidote for lead poisoning; however, wide inter- and intra-individual variation can be used (Bradberry et al., 2009; Package Insert, 2007). DMPS-DMSA can be alternated or replaced with DMPS (Bose-O'Reilly et al., 2003) at an exemplary dose of about 200-400 mg daily, depending on the clinical context.

In some embodiments, an iron chelator is administered to the patient, for example, as follows.

Deferasirox: Deferasirox can be administered at a dose of 20 to 40 mg/kg per day List, 2010). Deferiprone may be orally administered daily at a dose of about 40-90 mg/kg (Cermak et al., 2011; Cermak et al., 2013). Deferoxamine may be administered subcutaneously (s.c.), intravenously (i.v.), or intramuscularly (i.m.) at a dose of about 1-2 g, with dosing up to about 5-7 days or if given s.c., it can be infused s.c. overnight, (s.c. may be performed up to 5-7 nights per wk). Dexrazoxane may be combined with anthracyclines (e.g. idarubicin) to prevent cardiotoxicity during the induction, consolidation phase of therapy. For pediatric or adult leukemia, dexrazoxane may be combined with idarubicin for cardioprotection (Vachhani et al., 2017; Schloemer et al., 2017; Walker et al., 2013; Woodlock et al., 1998). Dexrazoxane can also be given with antioxidants/mineral/vitamins and low doses of anthracycline (e.g. idarubicin) maintenance phase of therapy. Dexrazoxane is given with idarubicin as follows or per package insert (Dexrazoxane, 2014). When given with idarubicin, dexrazoxane can be administered daily with Idarubicin as follows: dexrazoxane can be dosed at 50:1 (e.g. 500 mg/m² for Idarubicin dose of 10 mg/m²) ratio of dexrazoxane: idarubicin i.v. 30-min prior to each dose of Idarubicin.

N-Acetyl-Cysteine: N-Acetyl-Cysteine (NAC) can be administered, e.g., i.v. at a dose of about 200 mg/mL acetylcysteine (Acetadote). N-Acetyl-Cysteine may be orally administered, e.g., at a dose of about 600 mg NAC tablets 2× per day on the days that the patient did not receive the i.v. NAC.

In some embodiments, a copper chelator is administered to the patient, for example, as follows.

Trientine: The daily dose of trientine is preferably adjusted based on clinical response or toxicity (Fu et al., 2012). An increase in dose may be necessary if the free serum copper is persistently above 15 micrograms/milliliter (Prod Info Syprine(R), 1998). Maintenance doses should be monitored initially weekly and then at 1-3 month intervals or per discretion of physician. For example, oral trientine can be administered at a dose of about 500 mg, 4 times a day (2 times with meals, and 2 times without meals) initially, with dose adjustments as necessary to maintain serum ceruloplasmin levels at 5-15 mg/dL.

Trientine is can be given on an empty stomach, at least one hour before or 2 hours after a meal, and one hour before or after all drugs (Prod Info Syprine(R), 1998). The capsules should be swallowed whole with water and are normally not to be opened or chewed.

Tetrathiomolybdate (TM): Initial TM dosing may be about 180 mg of TM daily in 4 divided doses until copper levels decreased to a target range of 5-15 mg/dL (Chan et al., 2012). When copper levels are within a target of 5-15 mg/dL, patients are then switched to a lower dose of TM at 100 mg daily in divided doses.

Amifostine: Amifostine is a cytoprotective adjuvant that may be administered in a cancer therapy. Amifostine may be given prior to chemotherapy for bone marrow diseases, bone marrow failure, myeloproliferative neoplasms, MDA and/or MPN, AML, or ALL, on certain embodiments AML or ALL, to prevent toxicity (e.g. mucositis, organ toxicity) during low-intensity or high intensity regiments and during maintenance phase if needed to prevent toxicity.

Repurposing Amifostine may include but is not limited to the following: free-radical scavenging therapy with Amifostine as cancer and leukemia prevention; free-radical scavenging therapy with Amifostine as maintenance therapy for cancer/leukemia; or free-radical scavenging therapy with Amifostine for treatment and prevention of chronic diseases. Doses up to 1300 mg/m² may be administered about 15-30 minutes prior to chemotherapy or without chemotherapy.

Other diseases that may be treated with the present metal chelation drugs alone or in combination with antioxidants, minerals, and/or vitamins include, but are not limited to, neurologic disease, (e.g., Parkinson's and Alzheimer's), hematologic disease (e.g., aplastic anemia and myeloproliferative neoplasm), autoimmune/rheumatologic disease (e.g., rheumatoid arthritis and systemic lupus erythematosus (SLE)), infectious disease (e.g. fungal infections), renal disease (e.g., renal impairment, acute tubular necrosis, and acute renal failure), immunologic disease (immune deficiencies), psychiatric, ADHD, autism, musculoskeletal, dermatologic, ocular (e.g., macular degeneration), hepatic disease (e.g., hepatic steatosis), and gastrointestinal disease.

In another embodiment, methods for the treatment of prevention of osteoporosis is provided by administering metal chelation agents, antioxidants, minerals, vitamins, and/or free radical scavenging agents.

Further embodiments concern methods for correlating metallomic profiles with molecular, genomic, proteomic, and/or immunologic profiles. Subjects may be tested for metal content and metal transporter status during medical assessment of any patient seeking medical care including healthy patients, cancer and leukemia patients, and patients with acute and chronic diseases. Subjects may be assessed for nutritional status, microbiome status, trace mineral status, and/or oxidative stress status/anti-oxidant status by a panel during cancer treatment or for disease prevention. The subject may be monitored for environmental exposure (including metal values) during the course of cancer treatment or general medical follow-up (for, e.g., risk assessment) and intervention on the presence of toxic exposures may be given, such as counseling on the elimination of mattresses that contain toxic chemicals.

The methods described herein are useful in treating cancer. Generally, the terms “cancer” and “cancerous” refer to or describe the physiological condition in mammals that is typically characterized by unregulated cell growth. More specifically, cancers that are treated in connection with the methods provided herein include, but are not limited to, solid tumors, hematologic malignancies, metastatic cancers, or non-metastatic cancers. In certain embodiments, the cancer may originate in the lung, kidney, bladder, blood, bone, bone marrow, brain, breast, colon, esophagus, duodenum, small intestine, large intestine, colon, rectum, anus, gum, head, liver, nasopharynx, neck, ovary, pancreas, prostate, skin, stomach, testis, tongue, or uterus.

The cancer or related condition may specifically be of the following histological type, though it is not limited to these: neoplasm, malignant; carcinoma; non-small cell lung cancer; renal cancer; renal cell carcinoma; clear cell renal cell carcinoma; lymphoma; blastoma; sarcoma; carcinoma, undifferentiated; meningioma; brain cancer; oropharyngeal cancer; nasopharyngeal cancer; biliary cancer; pheochromocytoma; pancreatic islet cell cancer; Li-Fraumeni tumor; thyroid cancer; parathyroid cancer; pituitary tumor; adrenal gland tumor; osteogenic sarcoma tumor; neuroendocrine tumor; breast cancer; lung cancer; head and neck cancer; prostate cancer; esophageal cancer; tracheal cancer; liver cancer; bladder cancer; stomach cancer; pancreatic cancer; ovarian cancer; uterine cancer; cervical cancer; testicular cancer; colon cancer; rectal cancer; skin cancer; giant and spindle cell carcinoma; small cell carcinoma; small cell lung cancer; papillary carcinoma; oral cancer; oropharyngeal cancer; nasopharyngeal cancer; respiratory cancer; urogenital cancer; squamous cell carcinoma; lymphoepithelial carcinoma; basal cell carcinoma; pilomatrix carcinoma; transitional cell carcinoma; papillary transitional cell carcinoma; adenocarcinoma; gastrointestinal cancer; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma with squamous metaplasia; thymoma, malignant; ovarian stromal tumor, malignant; thecoma, malignant; granulosa cell tumor, malignant; androblastoma, malignant; Sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; lentigo maligna melanoma; acral lentiginous melanoma; nodular melanoma; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; Mullerian mixed tumor; nephroblastoma; hepatoblastoma; carcinosarcoma; mesenchymoma, malignant; Brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; an endocrine or neuroendocrine cancer or hematopoietic cancer; pinealoma, malignant; chordoma; central or peripheral nervous system tissue cancer; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; B-cell lymphoma; malignant lymphoma; Hodgkin's disease; Hodgkin's; low grade/follicular non-Hodgkin's lymphoma; paragranuloma; malignant lymphoma, small lymphocytic; malignant lymphoma, large cell, diffuse; malignant lymphoma, follicular; mycosis fungoides; mantle cell lymphoma; Waldenstrom's macroglobulinemia; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; chronic lymphocytic leukemia (CLL); acute lymphoblastic leukemia (ALL); hairy cell leukemia; chronic myeloblastic leukemia; hereditary (equivalently, family or inherited) cancer syndromes including hereditary breast and ovarian cancer syndrome, Li-Fraumeni syndrome, Cowden syndrome, Sezary syndrome and other cutaneous lymphomas, and Lynch syndrome; bone marrow diseases; bone marrow failure; myelodysplastic syndrome; and/or myeloproliferative neoplasms.

Additional hematologic malignancies which may be treated include but are not limited to: multiple myeloma; acute and chronic leukemias including Acute Lymphocytic Leukemia (ALL), Chronic Lymphocytic Leukemia (CLL), Acute Myeloid Leukemia(CML), Chronic Myeloid Leukemia(CML), and large granular lymphocytic leukemia (LGL); myeloproliferative neoplasms (MPNs) such as myelofibrosis (MF, including primary and secondary), polycythemia vera (PV), and essential thrombocytopenia (ET); myelodysplastic syndrome (MDS); MDS/MPN; and lymphomas, including Hodgkin's lymphoma and non-Hodgkin's lymphoma (low, intermediate, and high grade).

Regarding neoplastic condition (e.g., cancer) treatment, depending on the stage of the neoplastic condition, neoplastic condition treatment involves one or a combination of the following therapies: surgery to remove the neoplastic tissue, radiation therapy, and chemotherapy. Other therapeutic regimens may be combined with the administration of the anticancer agents, e.g., therapeutic compositions and chemotherapeutic agents. For example, the patient to be treated with such anti-cancer agents may also receive radiation therapy and/or may undergo surgery.

In the case of non-small cell lung cancer, the patient may undergo surgery to remove cancerous tissue. Such surgery may be a pneumonectomy, lobectomy, segmentectomy, wedge resection, or sleeve resection.

The patient may undergo radiation treatment, such as external beam radiation therapy or brachytherapy. The patient may undergo radiofrequency ablation, which uses high-energy radio waves to heat the tumor and destroy cancer cells.

The patient may undergo treatment with pharmacologic or biologic anti-cancer drug therapy. Anti-cancer therapies include cytotoxic therapies, hormonal therapies, and targeted therapies.

Non-limiting examples of anti-cancer therapeutic agents include:
1) inhibitors or modulators of a protein involved in one or more of the DNA damage repair (DDR) pathways such as:
   a. PARP1/2, including, but not limited to: olaparib, niraparib, rucaparib;
   b. checkpoint kinase 1 (CHK1), including, but not limited to: UCN-01, AZD7762, PF477736, SCH900776, MK-8776, LY2603618, V158411, and EXEL-9844;
   c. checkpoint kinase 2 (CHK2), including, but not limited to: PV1019, NSC 109555, and VRX0466617;
   d. dual CHK1/CHK2, including, but not limited to: XL-844, AZD7762, and PF-473336;
   e. WEE1, including, but not limited to: MK-1775 and PD0166285;
   f. ATM, including, but not limited to KU-55933,
   g. DNA-dependent protein kinase, including, but not limited to NU7441 and M3814; and
   h. Additional proteins involved in DDR;
2) Inhibitors or modulators of one or more immune checkpoints, including, but not limited to:
   a. PD-1 inhibitors such as nivolumab (OPDIVO), pembrolizumab (KEYTRUDA), pidilizumab (CT-011), cemiplimab (LIBTAYO), spartalizumab (PDR001), camrelizumab (SHR1210), sintilimab (IBI308), tislelizumab (BGB-A317), toripalimab (JS 001), and AMP-224 (AMPLIMMUNE);
   b. PD-L1 inhibitors such as atezolizumab (TECENTRIQ), avelumab (Bavencio), durvalumab (Imfinzi), MPDL3280A (Tecentriq), BMS-936559, and MEDI4736;
   c. anti-CTLA-4 antibodies such as ipilimumab (YERVOY) and CP-675,206 (TREMELIMUMAB);
   d. inhibitors of T-cell immunoglobulin and mucin domain 3 (Tim-3);
   e. inhibitors of V-domain Ig suppressor of T cell activation (Vista);
   f. inhibitors of band T lymphocyte attenuator (BTLA);
   g. inhibitors of lymphocyte activation gene 3 (LAGS); and
   h. inhibitors of T cell immunoglobulin and immunoreceptor tyrosine-based inhibitory motif domain (TIGIT);
3) telomerase inhibitors or telomeric DNA binding compounds;
4) alkylating agents, including, but not limited to: chlorambucil (LEUKERAN), oxaliplatin (ELOXATIN), streptozocin (ZANOSAR), dacarbazine, ifosfamide, lomustine (CCNU), procarbazine (MATULAN), temozolomide (TEMODAR), and thiotepa;
5) DNA crosslinking agents, including, but not limited to: carmustine, chlorambucil (LEUKERAN), carboplatin (PARAPLATIN), cisplatin (PLATIN), busulfan (MYLERAN), melphalan (ALKERAN), mitomycin (MITOSOL), and cyclophosphamide (ENDOXAN);
6) anti-metabolites, including, but not limited to: cladribine (LEUSTATIN), cytarbine, (ARA-C), mercaptopurine (PURINETHOL), thioguanine, pentostatin (NIPENT), cytosine arabinoside (cytarabine, ARA-C), gemcitabine (GEMZAR), fluorouracil (5-FU, CARAC), capecitabine (XELODA), leucovorin (FUSILEV), methotrexate (RHEUMATREX), and raltitrexed;
7) antimitotics, which are often plant alkaloids and terpenoids, or derivates thereof including but not limited to: taxanes such as docetaxel (TAXITERE), paclitaxel (ABRAXANE, TAXOL), vinca alkaloids such as vincristine (ONCOVIN), vinblastine, vindesine, and vinorelbine (NAVELBINE);
8) topoisomerase inhibitors, including, but not limited to: amsacrine, camptothecin (CTP), genisten, irinotecan (CAMPTOSAR), topotecan (HYCAMTIN), doxorubicin (ADRIAMYCIN), daunorubicin (CERUBIDINE)and combinations thereof, such as daunorubicin-cytarabine (VYXEOS), epirubicin (ELLENCE), ICRF-193, teniposide (VUMON), mitoxantrone (NOVANTRONE), and etoposide (EPOSIN);
9) DNA replication inhibitors, including, but not limited to: fludarabine (FLUDARA), aphidicolin, ganciclovir, and cidofovir;
10) ribonucleoside diphosphate reductase inhibitors, including, but not limited to: hydroxyurea;
11) transcription inhibitors, including, but not limited to: actinomycin D (dactinomycin, COSMEGEN) and plicamycin (mithramycin);
12) DNA cleaving agents, including, but not limited to: bleomycin (BLENOXANE), idarubicin,
13) cytotoxic antibiotics, including, but not limited to: actinomycin D (dactinomycin, COSMEGEN),
14) aromatase inhibitors, including, but not limited to aminoglutethimide, anastrozole (ARIMIDEX), letrozole (FEMARA), vorozole (RIVIZOR), and exemestane (AROMASIN);
15) angiogenesis inhibitors, including, but not limited to: genistein, sunitinib (SUTENT), and bevacizumab (AVASTIN);
16) anti-steroids and anti-androgens, including, but not limited to aminoglutethimide (CYTADREN), bicalutamide (CASODEX), cyproterone, flutamide (EULEXIN), nilutamide(NILANDRON);
17) tyrosine kinase inhibitors, including, but not limited to imatinib (GLEEVEC), erlotinib (TARCEVA), lapatininb (TYKERB), sorafenib (NEXAVAR), and axitinib (INLYTA);
18) mTOR inhibitors, including, but not limited to: everolimus, temsirolimus (TORISEL), and sirolimus;
19) monoclonal antibodies, including, but not limited to: trastuzumab (HERCEPTIN) and rituximab (RITUXAN);
20) apoptosis inducers such as cordycepin;
21) protein synthesis inhibitors, including, but not limited to: clindamycin, chloramphenicol, streptomycin, anisomycin, and cycloheximide;
22) antidiabetics, including, but not limited to: metformin and phenformin;
23) antibiotics, including, but not limited to:
   a. tetracyclines, including, but not limited to: doxycycline;
   b. erythromycins, including, but not limited to: azithromycin;
   c. glycylglycines, including, but not limited to: tigecyline;
   d. antiparasitics, including, but not limited to: pyrvinium pamoate;
   e. beta-lactams, including, but not limited to the penicillins and cephalosporins;
   f. anthracycline antibiotics, including, but not limited to: daunorubicin and doxorubicin;
   g. other antibiotics, including, but not limited to: chloramphenicol, mitomycin C, and actinomycin;
24) antibody therapeutic agents, including, but not limited to: muromonab-CD3, infliximab (REMICADE), adalimumab (HUMIRA), omalizumab (XOLAIR), daclizumab (ZENAPAX), rituximab (RITUXAN), ibritumomab (ZEVALIN), tositumomab (BEXXAR), cetuximab (ERBITUX), trastuzumab (HERCEPTIN), ADCETRIS, alemtuzumab (CAMPATH-1H), Lym-1 (ONCOLYM), ipilimumab (YERVOY), vitaxin, bevacizumab (AVASTIN), and abciximab (REOPRO);
25) conjugated antibody therapeutics, such as gemtuzumab ozogamicin (MYLOTARG) and inotuzumab ozogamicin (BESPONSA);
26) targeted therapies other than those otherwise classified above, including BCL-2 targeted therapies (e.g., oblimersen, venetoclax, and navitoclax), FLT3 inhibitor (e.g. midostaurin and gilteritinib), a BCR-ABL tyrosine kinase inhibitor (e.g., imatinib, nilotinib, dasartinib, bosutinib, posatinib, and bafetinib), a JAK-2 inhibitor (e.g., fedratinib, gandotinib, and pacritinib), an IDH1 inhibitor (e.g., ivosidenib) or IDH2 inhibitor (e.g., enasidenib); and
27) other agents, such as Bacillus Calmette—Guérin (B-C-G) vaccine; buserelin (ETILAMIDE); chloroquine (ARALEN); clodronate, pamidronate, and other bisphosphonates; colchicine; demethoxyviridin; dichloroacetate; estramustine; filgrastim (NEUPOGEN); fludrocortisone (FLORINEF); goserelin (ZOLADEX); interferon; leucovorin; leuprolide (LUPRON); levamisole; lonidamine; mesna; metformin; mitotane (o,p′-DDD, LYSODREN); nocodazole; octreotide (SANDOSTATIN); perifosine; porfimer (particularly in combination with photo- and radiotherapy); suramin; tamoxifen; titanocene dichloride; tretinoin; anabolic steroids such as fluoxymesterone (HALOTESTIN); estrogens such as estradiol, diethylstilbestrol (DES), and dienestrol; progestins such as medroxyprogesterone acetate (MPA) and megestrol; and testosterone.

The patient may undergo chemotherapy with one or more of cisplatin, carboplatin, paclitaxel, albumin-bound paclitaxel, docetaxel, gemcitabine, vinorelbine, irinotecan, etoposide, vinblastine, and pemetrexed. In addition, bevacizumab, ramucirumab, or necitumuab may also be used. If the patient's cancer expresses an increased level of EGFR, then the patient may also be treated with erlotinib, afatinib, gefitinib, osimertinib, or dacomitinib. If the patient's cancer has an ALK gene rearrangement, then the patient may also be treated with crizotinib, ceritinib, alectinib, brigatinib, or lorlatinib. If the patient's cancer expresses an altered BRAF protein, then the patient may also be treated with dabrafenib or trametinib. In certain embodiments, the patient may be treated with one or more of mylotarg, cladribine, idarubicin, and cytarabine, the combination of all of which is known as “CLIA-M.” In certain embodiments, the patient may be treated with one or more of cladribine, idarubicin, and cytarabine, the combination of all of which is known as “CLIA.”

For the treatment of disease, the appropriate dosage of a therapeutic composition will depend on the type of disease to be treated, as defined above, the severity and course of the disease, previous therapy, the patient's clinical history and response to the agent, and the discretion of the physician. The agent may be suitably administered to the patient at one time or over a series of treatments.

D. Combination Therapies

The methods and compositions, including combination therapies, enhance the therapeutic or protective effect, and/or increase the therapeutic effect of another anti-cancer or anti-hyperproliferative therapy. Therapeutic and prophylactic methods and compositions can be provided in a combined amount effective to achieve the desired effect, such as the killing of a cancer cell and/or the inhibition of cellular hyperproliferation. A tissue, tumor, or cell can be contacted with one or more compositions or pharmacological formulation(s) comprising one or more of the agents or by contacting the tissue, tumor, and/or cell with two or more distinct compositions or formulations. Also, it is contemplated that such a combination therapy can be used in conjunction with radiotherapy, surgical therapy, or immunotherapy; or with a targeted therapy.

Administration in combination can include simultaneous administration of two or more agents in the same dosage form, simultaneous administration in separate dosage forms, and separate administration. That is, the subject therapeutic composition and another therapeutic agent can be formulated together in the same dosage form and administered simultaneously. Alternatively, subject therapeutic composition and another therapeutic agent can be simultaneously administered, wherein both the agents are present in separate formulations. In another alternative, the therapeutic agent can be administered just followed by the other therapeutic agent or vice versa. In the separate administration protocol, the subject therapeutic composition and another therapeutic agent may be administered a few minutes apart, or a few hours apart, or a few days apart.

An anti-cancer first treatment may be administered before, during, after, or in various combinations relative to a second anti-cancer treatment. The administrations may be in intervals ranging from concurrently to minutes to days to weeks. In embodiments where the first treatment is provided to a patient separately from the second treatment, one would generally ensure that a significant period of time did not expire between the time of each delivery, such that the two compounds would still be able to exert an advantageously combined effect on the patient. In such instances, it is contemplated that one may provide a patient with the first therapy and the second therapy within about 12 to 24 or 72 h of each other and, more particularly, within about 6-12 h of each other. In some situations, it may be desirable to extend the time period for treatment significantly where several days (2, 3, 4, 5, 6, or 7) to several weeks (1, 2, 3, 4, 5, 6, 7, or 8) lapse between respective administrations.

In certain embodiments, a course of treatment will last 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

In some embodiments, a metal chelator can be administered concurrently with an anti-cancer therapy. In some embodiments, a metal chelator can be administered sequentially in combination with an anti-cancer therapy. Various combinations may be employed. For the example below a combination of a metal chelator is “A” and another anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present invention to a patient will follow general protocols for the administration of such compounds, taking into account the toxicity, if any, of the agents. Therefore, in some embodiments there is a step of monitoring toxicity that is attributable to combination therapy.

Combination therapies may comprise one or more of chemotherapy, epigenetic therapy, immunotherapy, and targeted therapy.

The chemotherapy may be induction chemotherapy and/or consolidation chemotherapy once a remission has been achieved. For example, metal chelation may be combined with an antioxidant, mineral, and/or vitamin along with chemotherapy, such as an anthracycline during induction or consolidation therapy, such as for AML. The chemotherapy may be high-intensity chemotherapy, such as idarubicin, cytarabine, or a nucleoside analog, such as fludarabine, clorfarabine, or cladribine. Targeted therapy can comprise a FLT3 inhibitor, BCR-ABL tyrosine kinase inhibitor, or a JAK-2 inhibitor.

Another combination, such as for ALL, may comprise metal chelation and an antioxidant, mineral, and/or vitamin in combination with chemotherapy, such as an anthracycline. The additional therapy may be hyper-CVAD chemotherapy comprising cyclophosphamide, vincristine, doxorubicin, and dexamethasone. The combination therapy may comprise an immunotherapy, such as rituximab, ofatumumab, blinatumomab, or other monoclonal antibody. The combination therapy can comprise an augmented Berlin-Frankfurt-Munster (BFM) chemotherapy.

The chemotherapy, such as for the treatment of AML, may be a low-intensity chemotherapy, such as a low dose of anthracycline in combination with low intensity AML therapy, such as hypomethylating therapy (e.g., decitabine or azacytidine), low-dose cytarabine, low-dose clofarabine, or low-dose cladribine. Targeted therapy can comprise a FLT3 inhibitor, BCR-ABL tyrosine kinase inhibitor, or a JAK-2 inhibitor.

The combination therapy, such as for the treatment of ALL, may comprise metal chelation and an antioxidant, mineral, or vitamin in combination with low-intensity chemotherapy, such as hyper-CVAD chemotherapy, or immunotherapy. The immunotherapy may be rituximab, ofatumumab, or blinatumomab.

For maintenance therapy, such as to prevent the relapse of AML or MDS, metal chelation may be administered alone or in combination with an antioxidant, mineral or vitamin and/or low-dose chemotherapy. Other combination agents may comprise a hypomethylator, immunotherapy, low-dose cytarabine, or a targeted therapy. POMP combination therapy chemotherapy may be used in the combination therapy.

Another component of the present combination therapy may be amifostine. Amifostine may be administered to prevent toxicity. Doses of amifostine up to 1300 mg/m² may be given over 15 minutes, 30 minutes prior to chemotherapy or without chemotherapy.

### II. PHARMACEUTICAL COMPOSITIONS

Pharmaceutical compositions of the present invention comprise an effective amount of one or more compounds of the present invention, e.g., a metal chelator, vitamin, mineral, and/or chemotherapeutic, or additional agent dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains at least one therapeutic compound or additional active ingredient will be known to those of skill in the art in light of the present disclosure, as exemplified by Remington: The Science and Practice of Pharmacy, 21^{st} Ed., Lippincott Williams and Wilkins, 2005, incorporated herein by reference. Moreover, for animal (e.g., human) administration, it will be understood that preparations should typically meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The therapeutic compounds described herein may comprise different types of carriers depending on whether it is to be administered in solid, liquid or aerosol form, and whether it need to be sterile for such routes of administration as injection. The present invention can be administered intravenously, intradermally, transdermally, intrathecally, intraarterially, intraarticularly, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The therapeutic compounds may be formulated into a composition in a free base, neutral or salt form, or in a complex form (e.g., a metal-ligand complex such as Ca-EDTA or Zn-dexrazoxane). Pharmaceutically acceptable salts include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present invention, the composition of the present invention suitable for administration is provided in a pharmaceutically acceptable carrier with or without an inert diluent. In some embodiments the composition is provided as a nanoformulation. The carrier should be assimilable and includes liquid, semi-solid, i.e., pastes, or solid carriers. Except insofar as any conventional media, agent, diluent or carrier is detrimental to the recipient or to the therapeutic effectiveness of the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in the composition include buffers, amino acids such as glycine and lysine, carbohydrates such as dextrose, mannose, galactose, fructose, lactose, sucrose, maltose, sorbitol, mannitol, etc.

In further embodiments, the present invention may concern the use of a pharmaceutical lipid vehicle compositions that include a therapeutic compound as described herein, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man) However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the therapeutic compound may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The composition may comprise one or more amphipathic molecules and/or emulsifying agents (e.g., lecithin). The dispersion may or may not result in the formation of liposomes.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

A. Alimentary Compositions and Formulations

In some embodiments, a therapeutic compound (e.g., vitamins, minerals, and/or a chelator) is administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

B. Parenteral Compositions and Formulations

In further embodiments, a compound or of the present invention may be administered via a parenteral route. For example, in some embodiments, it may be desirable to administer 2, 3, or 4 therapeutic compounds in the same pharmaceutical formulation to the patient to treat a cancer. As used herein, the term “parenteral” includes routes that bypass the alimentary tract. Specifically, the pharmaceutical compositions disclosed herein may be administered for example, but not limited to intravenously, intradermally, intramuscularly, intraarterially, intraarticularly, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). Generally, the solution is preferably sterile and fluid to the extent that easy injectability exists. It is preferably stable under the conditions of manufacture and storage and should be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. While, in some embodiments, fixed ratio combinations of chelators, vitamins, and minerals may be administered to a subject, the person responsible for administration may nonetheless make the final determination of the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

C. Miscellaneous Pharmaceutical Compositions and Formulations

In some embodiments, the active compounds or combination therapy may be formulated for administration via various miscellaneous routes, for example, topical or transdermal administration, mucosal administration (intranasal, vaginal, etc.) and/or inhalation. In some embodiments, the antioxidants and/or minerals (e.g., zinc, selenium, magnesium, and vitamin C) may be administered orally, and the iron chelator may be administered orally or intravenously.

### III. PHARMACEUTICAL COMBINATIONS

Also disclosed herein are pharmaceutical combinations, i.e., kits. A kit may comprise, in a single package, at least one pharmaceutically active ingredient together with instructions for use and optionally, one or more other active agent(s). For example, in some embodiments disclosed herein, a pharmaceutical combination comprises at least one anti-cancer pharmacologic therapy and one or more metal chelators, and optionally, at least one antioxidant, vitamin, or essential mineral. In other embodiments disclosed herein, a pharmaceutical combination comprises one or more metal chelators and at least one antioxidant, vitamin, or essential mineral, and optionally, at least one anti-cancer pharmacologic therapy. The kits may instruct the administration of the agents concurrently or sequentially; the intent of the kit is that they be used together.

### IV. DEFINITIONS

As used herein, the terms below have the meanings indicated.

As used herein throughout the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

The phrase “therapeutically effective” is intended to qualify the amount of an active ingredient, or a combination of active ingredients, used in the treatment of a disease or disorder or on the effecting of a measurable parameter such as a clinical endpoint. An amount of a combination of active ingredients, for example a metal chelator and either an anti-cancer therapy or an antioxidant, or both, may be therapeutically effective where the individual active ingredients are not present in a therapeutically effective amount.

As used herein, the term “metal,” e.g. in the context of chelation, includes metal ions and any forms of a metal likely to be found in a mammalian body, such as metal salts, sequestered metal ions, bound metal ions, and the like.

As used herein, the terms “cancer therapy” and “anti-cancer therapy” are synonymous.

As used herein, the terms “anti-cancer pharmacologic therapy” and “chemotherapy” are synonymous.

The term “subject” or, equivalently, “patient” as used herein refers to any individual to which the subject methods are performed. Generally, the patient is human, although as will be appreciated by those in the art, the patient may be an animal. Thus, other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of patient.

“Treatment” and “treating” refer to administration or application of a therapeutic agent to a subject or performance of a procedure or modality on a subject for the purpose of obtaining a therapeutic benefit of a disease or health-related condition. For example, a treatment may include administration chemotherapy, immunotherapy, radiotherapy, performance of surgery, or any combination thereof.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the invasiveness of a tumor, reduction in the growth rate of the cancer, or prevention of metastasis. Treatment of cancer may also refer to prolonging survival of a subject with cancer. Treatment of cancer may also refer to reduction in tumor burden. Additional measures relevant to treatment of cancer are given herein, such as overall survival, remission, time to disease progression, and the like.

Likewise, an effective response of a patient or a patient's “responsiveness” to treatment refers to the clinical or therapeutic benefit imparted to a patient at risk for, or suffering from, a disease or disorder. Such benefit may include cellular or biological responses, a complete response, a partial response, a stable disease (without progression or relapse), or a response with a later relapse. For example, an effective response can be reduced tumor size or progression-free survival in a patient diagnosed with cancer.

As used herein, a “broad-spectrum” metal chelator is one that chelates more than one metal, and preferably, chelates potentially toxic, non-essential metals, e.g., those chosen from arsenic (As), aluminum (Al), antimony (Sb), barium (Ba), boron (B), cadmium (Cd), Cerium (Ce), Chromium (Cr), lead (Pb), mercury (Hg), neodymium (Nd), Nickel (Ni), tin (Sn), titanium (Ti), uranium (U), and vanadium (V). In some embodiments, however, these broad-spectrum chelators will also chelate Iron (Fe), copper (Cu), and the like.

As used herein, a “targeted therapy” may refer to i) a therapy targeting a particular class of molecules involved in tumor development or oncogenic signaling, for example an antibody, or ii) a means of delivery that brings a therapeutic molecule such as an antibody or drug selectively or preferentially to the target cells or tissue.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Metal Chelation Therapy for Treatment of Disease

Some metals can induce dose-dependent chromosomal breaks, gaps and fragments. Zinc is found in over 300 metalloenzymes and impacts gene expression by a variety of ways including structural stability and functionality of a variety of transcription factors as well as tumor suppressors (VanLandingham et al., 2002; Ostrakhovitch and Cherian, 2005). In this study, the effect of broad-spectrum metal detoxification and minerals, vitamins, and/or antioxidants on genetic stability was evaluated.

Methods. Approximately two milliliters of serum, approximately two milliliters of BM aspirate, and approximately 1-2 millimeters of each core BM biopsy were obtained from leukemia patients and controls who were receiving bone marrow biopsies and blood draws or biologic sample collection for other purposes. Serum samples were digested and mineralized in perfluoroalkoxy vessels at about 90° C. with HNO₃ and H₂O₂ and then evaporated in concentrated HCl at about 130° C. under a laminar flow hood.

Stable dried specimens were analyzed for trace metal content using inductively coupled plasma mass spectroscopy (ICP-MS). Dry specimens were taken up in one mL 10% H₃NO₂, then diluted to a volume of 10 mL. One microliter of this solution is injected into the analyzer using an auto sampler, where the analyte is ionized in hot argon plasma. The ions are then separated using a magnetic sector mass analyzer (quadrupole analyzer) and their intensities monitored. Arsenic (As) suffers from molecular interferences (argon chloride dimers), and required operating the instrument at higher mass resolution mode. Analyses were reported as metal concentrations relative to Ca and/or P, e.g., Hg/Ca, because of the effects of varying bone density due to porosity and predominant tissue types. Metal concentrations normalized to Ca content can be obtained with high relative precision (<3% of 1 SD) and accuracy (<10% of 1 SD).

p53 mutational status was assessed using standard of care including next generation sequencing.

Subjects. Subject characteristics are given below in Table 1.

TABLE 1
Subject Characteristics

| Median Age Serum | | Median Age BM | |
|---|---|---|---|
| Patient | Control | Patient | Control |
| N = 200 | N = 92 | N = 24 | N = 13 |
| N(%) | N(%) | N(%) | N(%) |
| 58 (18-88) | 51 (23-82) | 68 (29-88) | 41 (26-62) |

Results. It was observed that metal levels vary significantly between cytogenetic risk groups in AML. Cytogenetic-risk groups in patients with newly diagnosed AML were found to be significantly associated with differences in copper (Cu) and zinc levels (Table 2). Studies also showed that p53 mutational status was significantly associated with differences in copper, zinc, and arsenic levels (Table 3). Therefore, the procedure of metal detoxification plus administration of antioxidants, minerals, and/or vitamins would be expected to normalize the abnormalities and, thereby, restore genetic stability. These observations also demonstrate that metals can be utilized for risk assessment during treatment just like cytogenetic and molecular mutations are already used for prognostication.

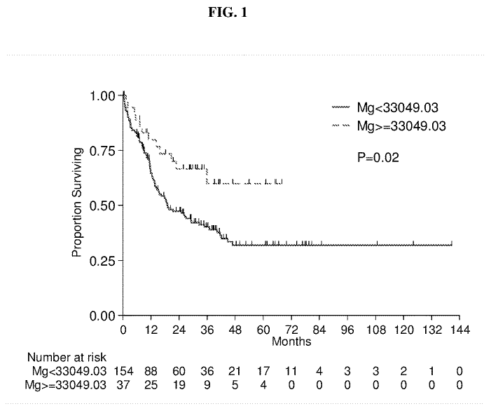

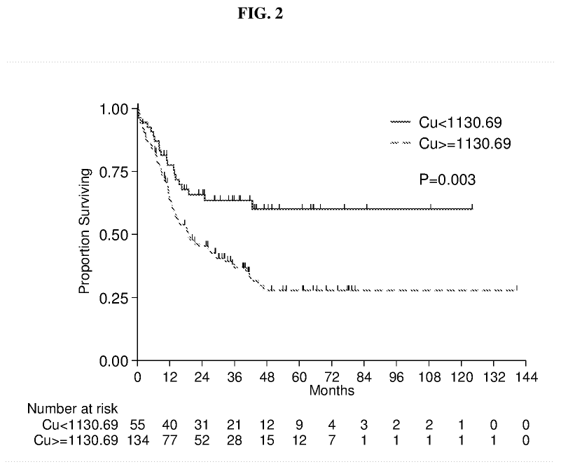

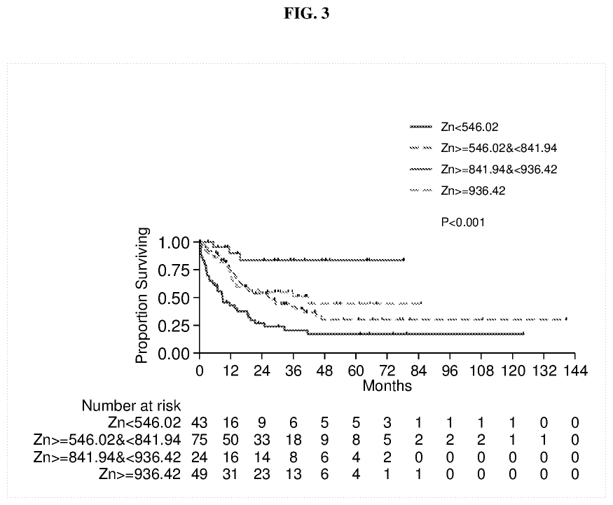

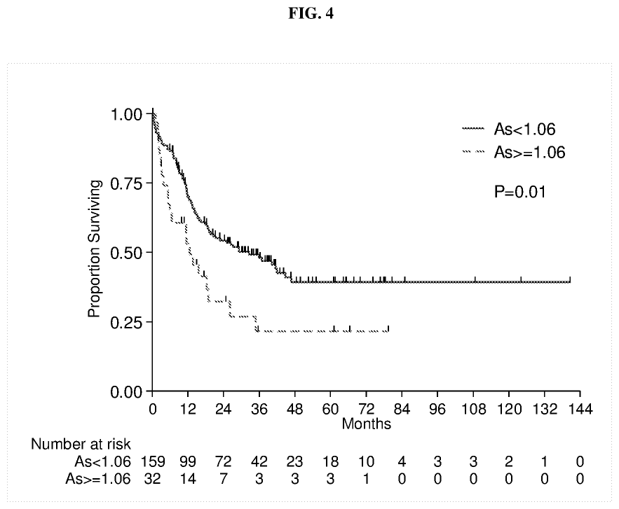

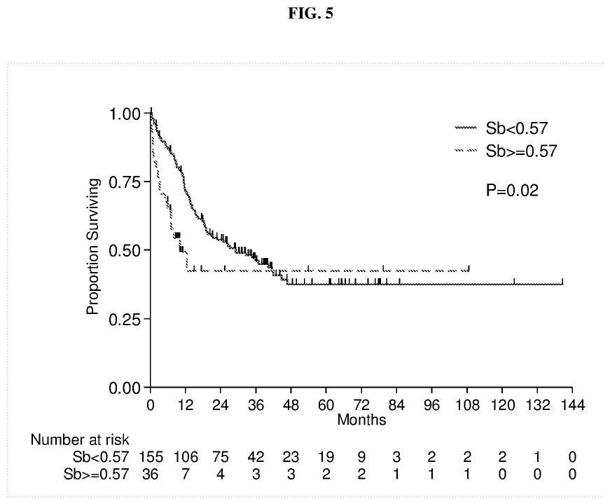

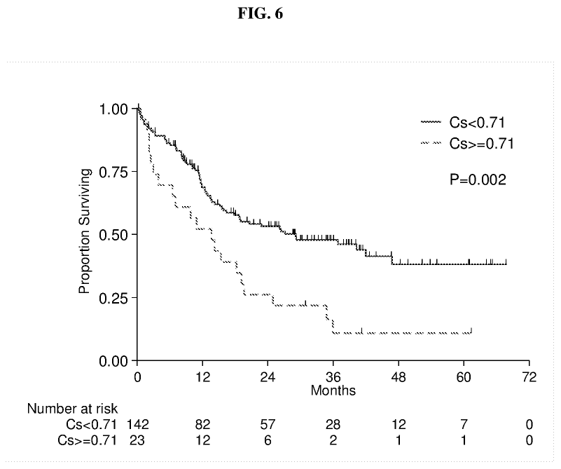

It was observed that toxic and essential metal levels vary significantly in the bone marrow and serum of normal individuals compared to patients with acute myeloid leukemia (AML). For example, the bone marrow of patients with AML contained significantly higher levels cadmium, lead, antimony, and arsenic (Table 4). The serum contains significantly elevated levels of toxic metals such as antimony, cadmium, mercury, arsenic, and uranium and significantly lower levels of certain essential metals such as iodine and selenium, whereas the essential elements copper and iron were significantly elevated in AML compared to normal controls. Overall survival in patients with AML was significantly worse in patients with higher levels of certain toxic metals (e.g. mercury, antimony and lead). As shown in FIGS. 1-6, lower levels of certain essential metals (e.g. magnesium and zinc) were associated with inferior survival and excess levels of the essential element copper were associated with inferior survival. Thus, metal values are useful for risk-stratification during cancer and leukemia treatment, and unfavorable metal profiles may be modified in cancer patients in order to improve outcomes. These observations provide rationale for metal detoxification and rebalancing of minerals and antioxidants to eliminate excess levels of certain toxic metals while replacing mineral/antioxidant deficiencies. Results are also shown in FIGS. 1-6.

Metal levels were measured in biological samples from patients with AML and control samples.

Results are provided below in Tables 2-5. Statistically significant differences were observed. As shown in Table 3, p53 mutational status was significantly associated with levels of copper, zinc, and arsenic. Values are given below in median and (range).

TABLE 2
Cytogenetic-risk groups in AML are significantly associated with differences in copper (Cu)
and zinc levels.

| Serum Metal | Adverse | Intermediate | Favorable | |
|---|---|---|---|---|
| Level (ppb) | (n = 60) | (n = 114) | (n = 15) | P-value |
| Copper | 1,565 (646-3,424) | 1,295 (587-2,311) | 1,011 (269-2,384) | 0.001 |
| Zinc | 602 (215-3,396) | 788 (311-3,248) | 841 (271-1,802) | 0.003 |

TABLE 3
p53 mutational status is significantly associated with
copper, zinc, and arsenic levels.

| Serum Metal | Mutated p53 | Wild type p53 | |
|---|---|---|---|
| (ppb) | (n = 20) | (n = 117) | P-value |
| Copper ppb | 1,521 (995-2,224) | 1,295 (269-2,985) | 0.036 |
| Zinc ppb | 498 (260-1,011) | 830 (215-3,396) | <0.001 |
| Arsenic ppb | 0.714 (0-5) | 0 (0-22) | <0.001 |

TABLE 4
Bone Marrow

| | Patients (N = 24) | Control (N = 13) | |
|---|---|---|---|
| Metal | median (min-max) | median (min-max) | P-value |
| B | 592.96 (0.45-2,901.96) | 286.32 (0-623.44) | 0.01 |
| Ti | 1,458.03 (16.67-7,251.61) | 623.97 (259.32-1,657.74) | 0.01 |
| V | 1,209.95 (9.34-6,001.44) | 370.09 (56.32-1,793.88) | 0.004 |
| As | 170.61 (1.13-734.68) | 60.90 (26.21-240.70) | 0.01 |
| Se | 1,647.45 (24.96-8,325.92) | 865.68 (0-2, 403.31) | 0.02 |
| Cd | 15.55 (0.25-88.36) | 6.13 (1.11-40.55) | 0.04 |
| Sn | 1,017.77 (13.63-11,485.73) | 236.83 (68.89-1,744.39) | 0.01 |
| Sb | 64.37 (0-229.26) | 14.93 (0-115.48) | 0.01 |
| Nd | 15.32 (0-82.86) | 5.06 (1.75-12.90) | 0.01 |
| Pb | 482.07 (8.58-2,077.93) | 210.76 (60.38-423.55) | 0.004 |

TABLE 5
Serum

| | Patients (N = 200) | Control (N = 92) | |
|---|---|---|---|
| Metal | median (min-max) | median (min-max) | P-value |
| Li | 0.66 (0-44.22) | 1.24 (0.23-7.91) | <0.001 |
| B | 77.58 (1.64-572.84) | 58.72 (39.50-206.32) | 0.02 |
| Mg | 27,925.72 (9,229.32-99,627.72) | 21,905.15 (15,986.47-29,358.33) | <0.001 |
| Al | 19.33 (5.66-336.43) | 1.52 (0.28-14.48) | <0.001 |
| Ti | 87.44 (3.47-638.47) | 58.86 (43.75-69.89) | <0.001 |
| V | 0 (0-102.52) | 0 (0-0) | <0.001 |
| Cr | 0.49 (0-65.12) | 0 (0-0.02) | <0.001 |
| Mn | 0.51 (0-21.63) | 0 (0-0.02) | <0.001 |
| Fe | 1,782.04 (347.00-7,370.91) | 1,394.85 (638.08-3,170.68) | <0.001 |
| Co | 0 (0-9.80) | 0.16 (0.11-0.76) | <0.001 |
| Ni | 1.02 (0-24.08) | 0 (0-8.88) | <0.001 |
| Cu | 1,347.97 (269.06-3,424.44) | 1,049.41(658.84-1,871.13) | <0.001 |
| Zn | 755.05 (215.43-3,395.58) | 767.13 (471.70-1,457.83) | 0.43 |
| Ga | 0 (0-9.74) | 0 (0-0) | <0.001 |
| Se | 147.53 (32.37-291.00) | 191.00 (138.40-272.80) | <0.001 |
| Rb | 203.34 (80.32-803.97) | 314.94 (100.63-643.18) | <0.001 |
| Sr | 41.93 (0-879.02) | 25.46 (10.93-56.40) | <0.001 |
| Mo | 0 (0-7.22) | 0.84 (0.25-2.90) | <0.001 |
| Cd | 0 (0-3.06) | 0 (0-0) | <0.001 |
| Sb | 0 (0-38.72) | 0.99 (0.77-2.57) | <0.001 |
| I | 190.72 (9.71-146,376.92) | 124.15 (71.17-231.35) | 0.002 |
| Ba | 60.92 (0-240.51) | 0 (0-0) | <0.001 |
| Ce | 7.94 (0-145.99) | 0 (0-0) | <0.001 |
| Nd | 0 (0-15.21) | 0 (0-0) | <0.001 |
| Au | 0 (0-370.85) | 0 (0-0) | 0.01 |
| Hg | 0.39 (0-8.28) | 0 (0-0.04) | <0.001 |
| Ti | 0 (0-5.13) | 0 (0-0) | <0.001 |
| Bi | 0 (0-5.54) | 0 (0-0.01) | <0.001 |
| U | 0 (0-0.88) | 0 (0-0) | <0.001 |

### Example 2

### Multi-Metal Scoring System in AML

A total of 67 patients with newly diagnosed AML and 94 healthy volunteers underwent serum collection for trace metal analysis . The median age was 67 years (range 28-87 years) for the AML patients and 58 years (range 20-89 years) for the control group. The two groups had similar distributions of males and females. Most patients with AML lacked FLT3 internal tandem duplication (ITD) mutations (75.6%) or mutations in NPM1 (68.1%) and were considered to have intermediate-risk cytogenetic profiles (63.1%).

For this case-control study, blood serum samples were collected from control participants with no hematologic malignancies and patients with newly diagnosed AML prior to initiation of front-line chemotherapy. The diagnosis of AML was confirmed via analysis of bone marrow morphology, and bone marrow aspirates and biopsies were obtained from all AML patients. All AML cases were sub-classified using the World Health Organization's 2016 AML classification criteria. To determine the immunophenotypic features of the samples, multicolor flow cytometry was performed as previously described. Conventional cytogenetic analysis was performed on G-banded metaphases from bone marrow aspirates cultured without mitogen stimulation using standard techniques and was reported using the International System for Human Cytogenetic Nomenclature. Molecular analysis was performed using a polymerase chain reaction-based assay, and fluorescence in situ hybridization analysis was performed on bone marrow cultures using a dual-color break-apart probe as previously described. Patients were risk-stratified according to karyotype, gene mutations and per ELN classification.

Serum samples were digested and mineralized in perfluoroalkoxy vessels at 130° C. with HNO₃ and H₂O₂ and then evaporated in concentrated HCl at 120° C. under a laminar flow hood. Stable dried specimens were analyzed for trace metal content using inductively coupled plasma mass spectroscopy (ICP-MS).

Results. Median levels of metals obtained from patients and controls are given below in Table 6.

TABLE 6

| | AML | | Control | | |
|---|---|---|---|---|---|
| Metal (units) | Median | Range | Median | Range | P-value |
| Pb (nmol/L) | 25.965 | 0.000-286.921 | 2.172 | 0.048-75.000 | <0.001 |
| Cd (nmol/L) | 0 | 0.000-5.160 | 0.089 | 0.089-6.316 | 0.27 |
| Se (μmol/L) | 1.196 | 0.000-2.76 | 2.558 | 0.205-5.633 | <0.001 |
| Ti (μmo1/L) | 0.311 | 0.000-1.049 | 0.067 | 0.000-4.030 | <0.001 |
| Mg (mmol/L) | 0.97 | 0.475-1.701 | 1.04 | 0.644-2.514 | 0.06 |
| Zn (μmol/L) | 17.461 | 7.377-135.462 | 14.907 | 8.602-23.238 | 0.001 |
| Cr (μmol/L) | 0.062 | 0.000-10.185 | 0.02 | 0.000-1.245 | 0.001 |
| Fe (μmo1/L) | 35.111 | 11.735-147.644 | 28.945 | 8.437-62.536 | <0.001 |
| Co (μmol/L) | 0.011 | 0.000-0.400 | 0.011 | 0.000-12.899 | 0.75 |
| Cu (μmol/L) | 19.494 | 6.939-37.963 | 16.406 | 11.292-39.272 | 0.16 |
| As (μmol/L) | 0.005 | 0.000-0.491 | 0.02 | 0.000-0.359 | 0.003 |
| Rb (μmol/L) | 2.993 | 1.233-7.624 | 3.186 | 1.253-6.927 | 0.64 |
| Sr (μmo1/L) | 0.349 | 0.094-1.589 | 0.372 | 0.156-5.323 | 0.58 |
| Ba (μmol/L) | 0.021 | 0.004-0.063 | 0.018 | 0.000-0.070 | 0.05 |
| Pt (nmol/L) | 0 | 0.000-2.768 | 0.051 | 0.051-2.255 | <0.001 |
| Au (nmol/L) | 0 | 0.000-19.851 | 0.051 | 0.051-89.406 | <0.001 |
| Ca (mmol/L) | 2.437 | 2.417-3.118 | 2.463 | 2.416-3.116 | <0.001 |
| Na (mmol/L) | 134.737 | 89.571-196.658 | 129.759 | 99.119-181.679 | 0.03 |

Metal Scoring. A novel metal scoring system was utilized to risk-stratify the patients. The scoring system was based on whether a patient's values for ten toxic and essential metals were above or below specified cutoff values. These cutoff values were determined based on a combination of clinical judgement and the serum metal value distributions in the control group. Patients were assigned one point for each metal that fell outside the “target range” (i.e. a metal value above or below the predetermined limits). The overall score was a sum of the points. The rationale for constructing the score is straightforward. Because relative deficiencies of calcium, magnesium, selenium, zinc, and rubidium are associated with malignancies, patients received one point for any value lower than the specified values in Table 7. Relative elevations of copper and iron also receive one point each because they have been associated with malignancies. Cadmium, lead, and arsenic are established carcinogens, and thus were also included in the scoring system, each receiving one point when exceeding the tabled limits in Table 6. For seven patients, calcium levels by ICPMS were not available for the score and standard of care calcium values obtained from the clinical laboratory were used.

TABLE 7
Metal Scoring

| Metal | Level | | Points |
|---|---|---|---|
| Ca | <2.432 | mmol/L | 1 |
| Mg | <1.069 | mmol/L | 1 |
| Se | <1.603 | μmol/L | 1 |
| Zn | <14.898 | μmol/L | 1 |
| Rb | <3.182 | μmol/L | 1 |
| Cd | >0.75 | nmol/L | 1 |
| Pb | >2.268 | nmol/L | 1 |
| Cu | >16.366 | μmol/L | 1 |
| Fe | >29.009 | μmol/L | 1 |
| As | >14.148 | nmol/L | 1 |

The scores may then be divided into risk groups. Risk groups may be stratified various ways, for example patients may be separated according to three groups representing low (1-3), medium (4-6) and high metal scores (7-10).

It should be understood by those of skill in the art that the cutoff values given above are examples, and that precise cutoff values for these groups depends on the size and other features of the patient group from whom they are drawn. Similarly, the assignment of risk groups may be made in alternate ways.

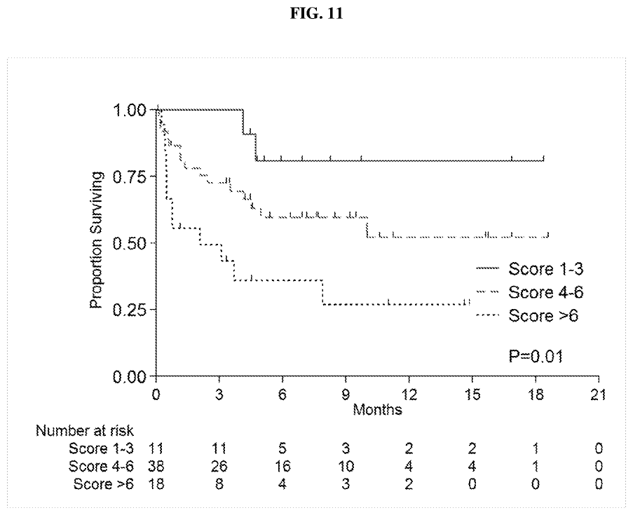

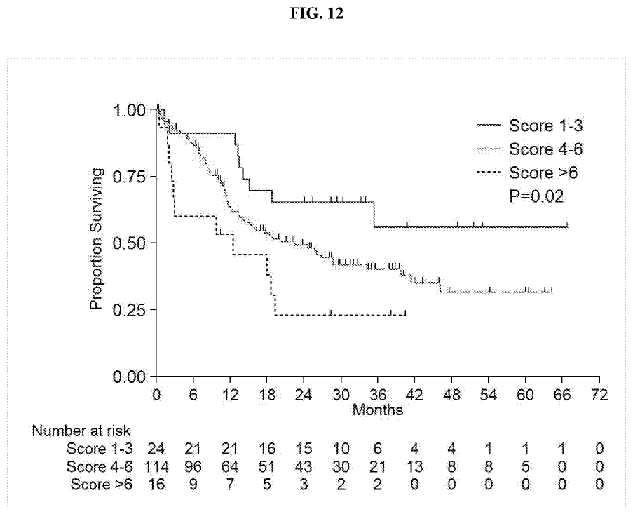

Results. Patients with higher metal scores did have significantly worse survival than patients with lower metal scores. FIG. 11 shows that in patients from this study, those with metal scores 1-3 had a 6-month survival estimate of 81% vs. 60% for scores 4-6 and 36% for scores 7-9 (p=0.01). FIG. 12 shows that in patients from a similar study at another hospital, those with metal scores 1-3 had a 6-month survival estimate of 91% vs. 87% for scores 4-6 and 60% for scores 7-9 (p=0.02).

These two analyses confirm that by stratifying patients by risk according to imbalance in their levels of essential and toxic metals, outcomes such as survival likelihood at a given date can be predicted. These data also support the notion that that rebalancing essential minerals and toxic metals in patients—essentially re-stratifying them to a lower-risk group—will improve their response to anti-cancer therapy.

### Example 3

### Positive Responses after Treatment with Metal Detoxification during AML and MDS Therapy

Clinical trials were performed—and continue—that utilize metal chelating agents plus antioxidants and minerals combined with standard therapy for myelodysplastic syndrome and AML. In one of the clinical trials the iron chelator dexrazoxane was used and in the other clinical trial Calcium-EDTA (Ca-EDTA) and/or DMSA was used. Demonstrably favorable early responses during AML/MDS treatment were observed when metal chelation and antioxidants/minerals were combined with standard AML or MDS therapy.

### Dexrazoxane in AML, High-Risk MDS, Myeloid Blast Phase of CML, Ph+AML, and Myeloid Blast Phase of MPNs

This ongoing study monitors the effect of 1) removal of toxic metals during AML, MPN, CML (myeloid blast phase), and therapy on improvement in clinical outcomes, 2) removal of toxic metals during MDS therapy on reduction in the rate transformation to AML from MDS, using dexrazoxane and antioxidants/minerals (zinc, selenium, magnesium, vitamin C) combined with standard chemotherapy (mylotarg, cladribine, idarubicin, and/or cytarabine (araC), (together, “CLIA-M”)). As per standard clinical practice, the protocol also allows for standard FDA approved targeted therapy when clinically indicated for common targeted mutations. Because the study is ongoing, past or present tense may be used below.

Study endpoints included:
overall survival;
overall response;
event-free survival;
remission duration;
rates of complete remission (CR)/complete remission with incomplete blood count recovery (CRi). Other efficacy endpoints of interest include overall response, overall survival, event-free survival and remission duration;
recurrence-free survival rate at 6 months (the recurrence-free survival rate at 6 months is a binary endpoint where the recurrence including death occurred within 6 months of treatment is considered as “recurrence event”);
assessment of the metal chelation effects of dexrazoxane combined with chemotherapy (mylotarg, cladribine, idarubicin, and cytarabine) by quantifying concentrations of toxic and essential metals in blood and bone marrow before and during treatment; and
description of the relationship between pretreatment patient/disease characteristics (including cytogenetic and molecular abnormalities) and clinical outcomes.

Patients. Includes subjects aged 12+ who are ambulatory and capable of self-care with:
diagnosis of AML, high risk MDS (>/=10% blasts or IPSS>/=intermediate-2), high-risk myeloproliferative neoplasm; patients with untreated or previously treated CML in myeloid blast phase or (Philadelphia Chromosome-positive (Ph+)) AML, isolated extramedullary myeloid neoplasm, and active CNS disease eligible;
Adequate organ function as defined below:
   liver function (bilirubin <2mg/dL, AST and/or ALT<3×ULN− or <5×ULN if related to leukemic involvement)
   kidney function (creatinine <1.5×ULN), unless due to leukemia/hematologic malignancy
      hyperbilirubinemia is allowed if considered due to Gilbert's hyperbilirubinemia
baseline left ventricular ejection fraction (LVEF) is greater than or equal to 50% by echocardiography (Echo) or MUGA scan;

The study excludes patients with active heart disease (defined as unstable coronary syndromes, unstable or severe angina, and/or recent myocardial infarction (MI) within 6 months), decompensated heart failure (HF), clinically significant arrhythmia, severe valvular disease, history of coronary artery disease (CAD), psychiatric illness that would limit compliance with study requirements documented hypersensitivity to any of the components of the chemotherapy program, and any other condition, including the presence of laboratory abnormalities, which judged by the investigator, places the patient at unacceptable risk, as well as women who were pregnant, and/or nursing, and/or of childbearing potential, unless they have a negative urine pregnancy test within 7 days and committed to continued abstinence from heterosexual intercourse or adoption of at least one highly effective method of contraception.

Treatments. Chemotherapy was administered as set forth below, in phases from induction to remission consolidation, to maintenance.

Induction. Patients receive 1 or 2 induction cycles of therapy with mylotarg, cladribine, idarubicin and cytarabine with dexrazoxane (optional dose reductions per standard guidelines). Dexrazoxane is administered daily with idarubicin in a 50:1 (e.g. 500 mg/m² for Idarubicin dose of 10 mg/m²) ratio of dexrazoxane: idarubicin, IV 30-min prior to each dose of idarubicin. Mylotarg, cladribine, idarubicin (with dexrazoxane) and cytarabine induction: mylotarg 3 mg/m² (maximum dose 4.5 mg) are given IV over 2 hours for up to two separate doses on day 1 and again on day 4 if deemed in the best interests of the patient (e.g. for refractory/relapsed or proliferative disease); cladribine at a dose of 5 mg/m²/day is given IV over approximately 1 to 2 hours daily on days 1-5 ; dexrazoxane at a dose of 500 mg/m2/day IV over 15-30 minutes on days 1-3, administered 30 minutes prior to idarubicin; idarubicin at a dose of 10 mg/m2/day IV over 30 minutes on days 1-3, initiated approximately 1-2 hours following the start of the cladribine infusion; and cytarabine at a dose of 1 gram/m²/day IV over 2 hours daily on days 1-5, initiated approximately 3-6 hours following the start of the cladribine infusion (fit patients aged 60 years or younger may be administered 2 grams/m²/day IV over 2 hours daily on days 1-5). In patients with a PS 2 or age >60 years, the number of days of cladribine and cytarabine may optionally be reduced to 4 days (cladribine 5 mg/m²/day on days 1-4, cytarabine 1 gram/m²/day IV over 2 hours daily on days 1-4) or 3 days (cladribine 5 mg/m2/day on days 1-3, cytarabine 1 gram/m²/day IV over 2 hours daily on days 1-3). Concomitantly, the number of days of idarubicin (with dexrazoxane) is reduced to 2 days (Idarubicin 10 mg/m²/day IV over 30 minutes on days 1-2).

Patients who have not achieved CR/CRi following one induction course can receive a second induction course to optimize response if possible. A second induction course is given at the same dose as the previous course or in a dose-reduced fashion if indicated.

Remission Consolidation. Patients achieving a CR or CRi may receive up to 6 additional cycles of consolidation therapy as follows. Mylotarg, Cladribine, Idarubicin and Cytarabine Consolidation: mylotarg 3 mg/m² (maximum dose 4.5 mg) is given IV over 2 hours on day 1 of every cycle; cladribine at a dose of 5 mg/m²/day is given IV over approximately 1 to 2 hours daily on days 1-3; dexrazoxane at a dose of 400 mg/m²/day IV over 15-30 minutes on days 1-2, administered approximately 30 minutes prior to idarubicin; idarubicin at a dose of 8 mg/m²/day IV over 30 minutes on days 1-2, initiated approximately 1-2 hours following the start of the cladribine infusion; and cytarabine at a dose of 0.75 gram/m²/day IV over 2 hours daily on days 1-3, initiated approximately 3-6 hours following the start of the cladribine infusion (fit patients aged 60 years or younger are optionally administered 1.5 grams/m²/day IV over 2 hours daily on days 1-3). In patients with a PS 2 or age >60 years, consolidation are optionally reduced to 2 days for all agents. Cycles are given approximately every 3-7 weeks upon recovery of counts and toxicities. One cycle of therapy is considered 4 weeks. Subsequent cycles may be started within 3-7 weeks after the start of the previous cycle depending on hematopoietic recovery and resolution of toxicities in the judgment of the treating physician. Subsequent cycle delay beyond 7 weeks may be allowed after discussion with the principal investigator and documentation of the discussion.

Maintenance Therapy. Monthly cycles are continued to complete a total of 24 cycles of therapy: mylotarg 3 mg/m² (maximum dose 4.5 mg) on day 1 given IV over 2 hours of the first maintenance cycle, then optionally repeated every 2-3 months on day 1; idarubicin 4 mg/m² IV over 30 minutes on Day 1; low dose cytarabine 10 mg/m² subcutaneously BID for 7 days on Days 1-7 (may be self-administered at home); dexrazoxane at 50:1 ratio of dexrazoxane: idarubicin (e.g. 200 mg/m² for idarubicin 4 mg/m²) IV over 15 minutes on Day 1, administered approximately 30 minutes prior to each dose of idarubicin.

Cycles of maintenance are repeated approximately every 3-7 weeks (for example, every 28-42 days) upon recovery of counts and toxicities. Subsequent cycle delay beyond 7 weeks may be allowed after discussion with the principal investigator and documentation of the discussion.

Patients who are deemed by the treating physician to be too deconditioned for continuation on standard induction/consolidation therapy or who experienced significant toxicities from consolidation may proceed directly to maintenance therapy for up 24 cycles of maintenance and can even continue for up to 32 cycles total on study if continuing to have clinical benefit and if tolerating therapy well. Patients who are suitable for transplant may undergo allogeneic transplant per the treating physician's discretion. Doses may be reduced as clinically indicated and after discussion with the principal investigator. Cytarabine can be omitted during maintenance cycles of therapy if considered in the best interests of the patient.

Other therapeutic agents may optionally be administered, such as targeted therapies, ruxolitinib, venetoclax, drugs for patients with FLT3 mutations (e.g., sorafenib, midostaurin, gilteritinib, drugs for Philadelphia chromosome negative disease (e.g., tyrosine kinase inhibitors), drugs for myelofibrosis and other myeloproliferative neoplasms (e.g., JAK2 inhibitors), drugs for patients with IDH2 mutations (e.g., enasidenib), drugs for patients with IDH1 mutations (e.g., ivosidenib), drugs for patients with RAS mutations (e.g., trametinib), Intrathecal prophylaxis with cytarabine for patients at risk of CNS disease, supportive therapies including amifostine, hematopoietic growth factors such as filgrastim/filgrastim-sndz or pegfilgrastim (GCSF).

Premedications/supportive care for induction, consolidation and maintenance chemotherapy may also be used, including ondansetron 8-16 mg IV prior to chemo (excluding subcutaneous cytarabine), methylprednisone 40 mg IV daily prior each cytarabine dose during induction and consolidation, prednisolone acetate 1% ophthalmic solution (2 drops in each eye 4 times daily at the same time as the cytarabine infusion and continued for at least 2 days after the last dose of cytarabine, during induction and consolidation).

Antioxidants, Minerals, and Vitamins. Because metal chelation can deplete the antioxidants and minerals zinc, selenium, vitamin C, and magnesium, these antioxidants and minerals are replaced during treatment to achieve metal rebalancing. Daily doses of the following antioxidant vitamins and minerals are given: zinc 50 mg monomethionine or zinc citrate), vitamin C 1000 mg optionally with dihydroquercetin (10 mg), selenium 200 mcg, L-selenomethionine sodium selenite 200 mcg with Vitamin E (as D-alpha tocopheryl succinate 20.1 mg), and magnesium (as magnesium citrate) 160 mg.

Assessments. Toxic and essential metals and copper isotopic abundance ratios are measured in bone marrow and blood serum of all patients prior to treatment (within 28 days) and at up to three follow-up time points at approximately 1 month +/−1 week, 3 months +/−2 weeks, and 6 months +/−2 weeks during therapy for hematologic malignancy plus chelation treatment. Approximately 5 ml blood is collected for metal analysis of serum at each time point. Approximately 1 mm bone marrow biopsy and/or approximately 400 microL of bone marrow aspirate is collected.

Metals assessed include but are not limited to lead (Pb), cadmium (Cd), mercury (Hg), nickel (Ni), cobalt (Co), chromium (Cr), aluminum (Al), arsenic (As), calcium (Ca), selenium (Se), magnesium (Mg), copper (Cu), and zinc (Zn). Whole blood serum (approx. 1 mL) and bone marrow (approximately 1 mm biopsy and/or approximately 400 microL aspirate) is frozen at −20 to −80 degrees Celsius and samples is sent in batches to the Geosciences Lab in Lyon on dry ice. An aliquot of each sample is digested and mineralized in PFA vessels at 130° C. with HNO₃ and H₂O₂ and evaporated in concentrated HCl at 120° C. in a laminar flow hood. Stable dried specimens are ready for analysis. Trace metal content is determined using inductively coupled plasma mass spectroscopy (ICP-MS).

Response criteria (e.g., for AML and myeloid blast phase of MPN) are equivalent to those used below.

Results. Results are given below in Table 8.

TABLE 8
Responses in Patients with AML and High-risk MDS Treated with Metal
Chelator Dexrazoxane + Antioxidants/Minerals and Standard Therapy
(CLIA or CLIA-M) (NCT03589729)

| | Number of Patients | |
|---|---|---|
| Disease Group | (Total n = 17) | Best Response |
| Front-line de novo AML | n = 5 | 5/5 (100%) CR |
| Secondary AML | n = 2 | ½ (50%) CRi |
| Salvage 1 AML | n = 4 | 4 /4 (100%) CR/CRi |
| Salvage 1 Extramedullary | n = 1 | CR of myeloid sarcoma; |
| Myeloid Sarcoma of | | neurologic improvement; |
| Scalp and Peripheral | | Inevaluable for complete |
| Nerves | | neurologic response due to |
| | | not following up. |
| Salvage 2 and higher | n = 4 | 1/4 (25%) CRi |
| AML (patients received | | |
| 2-6 prior lines of therapy) | | |
| High-risk MDS | n = 1 | Partial Response (PR) |

Additionally, among six front-line, secondary, or relapsed/refractory patients with AML who were treated on a clinical trial of dexrazoxane, zinc, selenium, magnesium, vitamin C and standard AML therapy of cladribine, idarubicin, cytarabine, and mylotarg (CLIA-M), 4/6 patients experienced demonstrably favorable responses within 1 cycle of therapy including complete remissions (n=2), complete remission with incomplete platelet recovery (n=1), and a morphologic leukemia-free state (n=1). Of the 2/6 patients who did not respond after one cycle, one patient had concurrent relapsed/refractory metastatic ovarian cancer, and had already failed treatment for her therapy-related MDS, before developing AML. The patient was also refractory to front-line AML therapy, as well as, the regimen of dexrazoxane, antioxidants/minerals, and standard chemotherapy (CLIA-M). The other patient was a very heavily pre-treated refractory AML patient who had already failed 6 lines of AML therapy before initiating the trial of dexrazoxane, zinc, selenium, magnesium, vitamin C and CLIA-M. The clinical course of these six cases treated with dexrazoxane, zinc, selenium, magnesium, vitamin C and CLIA-M are described below.

Positive response after 1 cycle of treatment was observed in 4/6 patients, as follows. Patients were treated with dexrazoxane, zinc, selenium, magnesium, vitamin C and the standard AML therapy of cladribine, idarubicin, cytarabine, and mylotarg (CLIA-M).

Patient 1: A 55-60-year old female who was diagnosed with hypocellular myelofibrosis (MF) developed secondary AML while on treatment for MF. Cytogenetics showed monosomy 7. Molecular studies by NGS revealed the following mutations: ASXL1, CEBPA, ETV6, and RUNX1. After 1 cycle with dexrazoxane, zinc, selenium, magnesium, vitamin C and CLIA-M, she achieved a morphologic leukemia-free state (MLFS) with all mutations becoming undetectable.

Patient 2: A 40-45-year old female with primary refractory AML and adverse cytogenetics, including the chromosomal translocation t(3;3)(q21;q26.2) and the adverse gene rearrangement by FISH (fluorescent in situ hybridization), MECOM (EVI1) presented after failing standard chemotherapy with daunorubicin and cytarabine (also known as “7+3”). She was treated with the regimen dexrazoxane, zinc, selenium, magnesium, vitamin C and CLIA-M. After 1 cycle with dexrazoxane, zinc, selenium, magnesium, vitamin C and CLIA-M, she achieved CR with normal neutrophils and partial recovery of platelets (CRi). The cytogenetic burden of the t(3;3) abnormality decreased from 6 metaphases of involvement to 3 metaphases. Similarly, follow-up FISH results showed that the burden of MECOM (EVI1) gene rearrangement dropped from 75/200 to 15/200. She later achieved CR.

Patient 3: A 55-60-year-old female with AML with normal karyotype and multiple molecular mutations (DNMT3A, FLT3-D835, IDH2, NMP1, and PTPN11) was treated with the regimen dexrazoxane, zinc, selenium, magnesium, vitamin C and CLIA-M, along with standard targeted agents for the FLT3-D835 (midostaurin) and IDH2 (enasidenib, IDHIFA) mutations. After 1 cycle of treatment, the patient was in CR.

Patient 4: A 55-60-year old male with AML was diagnosed with normal karyotype AML and these mutations: NOTCH1, IDH2, DNMT3A, and BRINP3 mutations. After 1 cycle with dexrazoxane, zinc, selenium, magnesium, vitamin C and CLIA-M, the patient was in in CR.

Patient 5: A 50-55-year old female with metastatic stage IV ovarian cancer developed high-risk therapy-related MDS. She failed MDS therapy and progressed to secondary, therapy-related AML. She had multiple adverse features including a complex karyotype and a P53 mutation. After failing front-line AML therapy, she was treated with the regimen dexrazoxane, zinc, selenium, magnesium, vitamin C and CLIA-M. Within 1 cycle, she experienced multiple infectious complications including disseminated fusarium of the skin, and eventually transitioned to comfort care. Her leukemia did not respond to the treatment.

Patient 6: A 70-75-year old male with progressive refractory AML initiated therapy after failing 6 prior lines of AML therapy. At the time of initiating the regimen of dexrazoxane, zinc, selenium, magnesium, vitamin C, CLIA-M, and standard of care enasidenib for his IDH2-mutation, his peripheral blood blasts were 94%. During cycle 1, his peripheral blood blasts dropped to as low as 50%. He also demonstrated significant improvement in extensive oral leukemic infiltration of his gingiva (a.k.a “gingival hyperplasia”). He did not undergo bone marrow assessment due to deciding to transition to hospice care.

### Ca-EDTA and DMSA in AML, High-Risk MDS, Myeloid Blast Phase of CML, Ph+AML, and Myeloid Blast Phase of MPNs (NCT03630991)

An ongoing study monitors the effect of 1) removal of toxic metals during AML therapy on improvement in clinical outcomes, 2) removal of toxic metals during MDS therapy on reduction in the rate transformation to AML from MDS, both using Calcium Disodium Edetate (EDTA) (Ca-EDTA) and Dimercaptosuccinic Acid (DMSA). Additional data from patients treated with Ca-EDTA in a manner similar to the method described is given below. Because the study is ongoing, past or present tense may be used below.

Study endpoints included:
complete remission (CR) rate and the 1-year overall survival (OS) rate in AML patients undergoing therapy combined with DMSA and/or Ca-EDTA;
CR rate, partial remission (PR) rate and 6-month cytogenetic response in MDS patients undergoing therapy combined with DMSA and/or Ca-EDTA;
overall survival in AML and MDS patients undergoing AML and MDS therapy combined with DMSA and/or Ca-EDTA;
remission duration in AML and MDS patients undergoing AML and MDS therapy combined with DMSA and Ca-EDTA;
toxic and essential metal levels during AML and MDS therapy combined with DMSA and/or Ca-EDTA;
reduction in metals in the bone marrow and blood of newly diagnosed AML and MDS patients undergoing metal detoxification combined with standard AML/MDS therapy;
safety profile in AML and MDS patients undergoing AML and MDS therapy combined with DMSA and/or Ca-EDTA;
correlation of degree of metal chelation with the degree of therapeutic response and minimal residual disease (MRD); and
progression rate in MDS patients.

Patients. Include adults aged 18+ who are ambulatory and capable of self-care with:
newly diagnosed (or untreated) AML with poor-risk cytogenetics, poor-risk molecular, or secondary AML (i.e. therapy-related or evolved from antecedent hematologic malignancy;
newly diagnosed (or untreated) myeloid blast phase of MPN (including myeloid blast phase of CML);
newly diagnosed (or untreated) high-risk, very-high risk or secondary MDS;
newly diagnosed (or untreated) MDS/MPN (regardless of cytogenetic/molecular status);
relapsed and/or refractory AML, MDS, MDS/MPN, myeloid blast phase of MPN (including myeloid blast phase of CML) who are either salvage 1 or salvage 2;
   who, if already being treated, are within first 3 cycles of front-line therapy and benefitting from therapy or are on salvage therapy.

The study excludes patients with uncontrolled inter-current illness, as well as women who were pregnant, and/or nursing, and/or of childbearing potential, unless they have a negative urine pregnancy test within 7 days and committed to continued abstinence from heterosexual intercourse or adoption of at least one highly effective method of contraception.

Treatment. Broad-spectrum metal detoxification and rebalancing comprised Ca-EDTA, DMSA, and daily oral antioxidants, vitamins, and minerals (including vitamin C, vitamin E, vitamin K, thiamin, riboflavin, niacin, pantothenic acid, vitamin B6, folate, vitamin B12, biotin, choline, magnesium, zinc, selenium, inositol, and/or rubidium). Detoxification/rebalancing is given with planned chemotherapy or other planned infusions 4 times per cycle. Any schedule facilitating the patient receiving 4 doses per cycle is allowed. For this study, a cycle is anticipated to occur in a period of approximately but not limited to 1 month (+/−1 week), which is the usual duration of a cycle of AML or MDS therapy, but may be longer depending on chemotherapy treatment delays or other circumstances. The combination of Ca-EDTA and DMSA has been widely used to provide optimal broad-spectrum metal detoxification.

Ca-EDTA is given 4 days per cycle with planned Leukemia/MDS treatments or other planned infusions for six cycles. For example, Ca-EDTA can be given beginning day 1 of planned chemotherapy and continued daily on days 2-4. Missed doses can be made up per discretion of the treating physician. One cycle consists of 4 doses of Ca-EDTA. The target dose of Ca-EDTA is 1 g/m². The intended starting dose is at the −1 dose level (0.75 g/m²). Ca-EDTA is prepared according to standard practice and given as an infusion IV over approximately 30 minutes in ˜250 cc of normal saline. Ca-EDTA is not co-administered with chemotherapy; it is before or after chemotherapy (preferably before).

DMSA is given daily for 8 days beginning day 1+/−3 days of MDS or AML. Other schedules are allowed during the treatment cycle, as long as 8 doses of DMSA are given during a cycle of MDS/AML therapy. The target dose is 500 mg (approx. 350 mg/m²). The intended starting dose is at the dose level −1 (up to a maximum of 375 mg, approx. 262.5 mg/m²). DMSA is given a minimum of 1 hour before or after oral leukemia therapy therapy/chemo.

Dose escalation may proceed as below, in the absence of dose-limiting toxicity (e.g., hypocalcemia, hypomagnesemia, zinc deficiency, copper deficiency, low ferritin, etc.):

TABLE 9

| Dose Level | Ca-EDTA | DMSA |
|---|---|---|
| −2 | 50% (0.5 g/m2) | To a maximum of 250 mg |
| | | (175 mg/m2) |
| −1 (starting dose) | 75% (0.75 g/m2) | To a maximum of 375 mg |
| | | (262.5 mg/m2) |
| 1 (target dose) | 100% (1 g/m2) | To a maximum of 500 mg |
| | | (350 mg/m2) |

Daily multivitamins and minerals to supplement anticipated losses during the chelation regimen are also administered, for 28 consecutive day +/−1 week, every cycle. An example of a suitable multivitamin/mineral supplement is given below in Table 10.

TABLE 10

| | Amount Per | | % Daily |
|---|---|---|---|
| | Serving | | Value |
| Vitamin C (as calcium ascorbate and ascorbic acid) | 5,000 | mg | 5,556% |
| Vitamin E (as mixed tocopherols) (from soy) (400 IU) | 268 | mg | 1,787% |
| Vitamin K (as Menaquinone-7) | 90 | mcg | 75% |
| Thiamin (as benfotiamine) | 5 | mg | 417% |
| Riboflavin (as riboflavin-5-phosphate) | 5 | mg | 385% |
| Niacin (as niacinamide) | 10 | mg | 63% |
| Pantothenic acid (as calcium pantothenate) | 25 | mg | 500% |
| Vitamin B6 (as pyridoxal-5-phosphate) | 5 | mg | 294% |
| Folate (as Quatrefolic L-5 methyltetrahydrofolate, glucosamine salt) | 400 | mcg DFE | 100% |
| Vitamin B12 (as methylcobalamin) | 50 | mcg | 2,083% |
| Biotin | 25 | mcg | 83% |
| Magnesium (as magnesium glyclinate) | 135 | mg | 32% |
| Zinc (as zinc gluconate) | 50 | mg | 455% |
| Selenium (as L-selenomethionine) | 200 | mcg | 364% |
| Rubidium (from vegetable culture) | 400 | mcg | * |

Assessments. Toxic and essential metals are measured in bone marrow and blood serum of all patients prior to treatment (within 28 days) and at up to three follow-up time points at approximately 1 month +/−1 week, 3 months +/−2 weeks, and 6 months +/−2 weeks during therapy for hematologic malignancy plus chelation treatment. Approximately 5 ml blood is collected for metal analysis of serum at each time point. Approximately bone 1 mm bone marrow biopsy and/or approximately 400 microL of bone marrow aspirate is collected.

Metals assessed include but are not limited to lead (Pb), cadmium (Cd), mercury (Hg), nickel (Ni), cobalt (Co), chromium (Cr), aluminum (Al), arsenic (As), calcium (Ca), selenium (Se), magnesium (Mg), manganese (Mn), copper (Cu), and zinc (Zn). Whole blood serum (approx. 1 mL) and bone marrow (approximately 1 mm biopsy and/or approximately 400 microL aspirate) is frozen at −20 to −80 degrees Celsius and samples is sent in batches to the Geosciences Lab in Lyon on dry ice. An aliquot of each sample is digested and mineralized in PFA vessels at 130° C. with HNO₃ and H₂O₂ and evaporated in concentrated HCl at 120° C. in a laminar flow hood. Stable dried specimens are ready for analysis. Trace metal content is determined using inductively coupled plasma mass spectroscopy (ICP-MS).

A NGS 300-gene panel test is also administered on BM aspirate (preferred) or blood from enrolled patients (approximately 3-5 mL for bone marrow or approximately 5-10 ml blood).

The metal reduction from baseline is compared between responders and non-responders. Two sample t-tests are used to compare the degree of metal chelation between the responders and non-responders to AML/MDS regimen+chelating agent DMSA and Ca-EDTA at each time point when metal level measurement is taken if the data are Normal and a Wilcoxon rank sum test otherwise. Correlation between metal level, gene mutations, and MRD status is assessed with t-test. Logistic regression is applied to examine the association between cytokine response and the change in essential metal level along with other clinical predictors. In addition, a linear mixed model is fitted to evaluate the difference in metal contents between responders and non-responders by using all measurements over time.

Response criteria for AML and myeloid blast phase of MPN. Response criteria for AML and myeloid blast phase of MPN are modified from the International Working Group for AML (JCO 2003; 21: 4642-9). Responders are patients who obtain a CR, CRi, or PR, with or without cytogenetic response, hematologic improvements, and morphologic leukemia-free state.

Overall survival (for AML, MPN, MDS, etc.) is defined as the time from treatment start till death or last follow-up. The distribution of time-to-event endpoints like overall survival etc. are estimated using the method of Kaplan and Meier. Comparisons of time-to-event endpoints by important subgroups (for example treatment time differences) is made using the log-rank tests.

Complete remission (CR) is defined as: clinical and/or radiologic evidence of disease becoming undetectable, including extramedullary leukemia; neutrophil count ≥1.0×10⁹/L and platelet count ≥100×10⁹/L, and bone marrow differential showing ≤5% blasts; peripheral blood counts showing no circulating blasts, neutrophil count ≥1.0×10⁹/L, platelet count ≥100×10⁹/L; and bone marrow aspirate and biopsy showing ≤5% blasts, no Auer rods, and no extramedullary leukemia.

Complete remission with incomplete blood count recovery (CRi) is defined as: having met all criteria for CR, except for either residual neutropenia (ANC <1.0×10⁹/L) or thrombocytopenia (platelet count <100×10⁹/L); peripheral blood counts showing no circulating blasts, neutrophil count <1.0×10⁹/L, and platelet count <100×10⁹/L; and bone marrow aspirate and biopsy showing <5% blasts, no Auer rods, and no extramedullary leukemia.

Partial Remission is defined as all CR criteria if abnormal before treatment except ≥50% reduction in bone marrow blast but still >5%.

A morphologic leukemia-free state is defined as having bone marrow with ≤5% myeloblasts.

A hematologic improvement (HI) is described by the number of positively affected cell lines:
erythroid response (E) (pretreatment Hgb <11 g/dL): Hgb increase by 1.5 g/dL
platelet response (P) (pretreatment platelets <100×10⁹/L):
   absolute increase of ≥30×10⁹/L for patients starting with >20×10⁹/L platelets
   increase from <20×10⁹/L to >20×10⁹/L and by at least 100%
neutrophil response (N) (pretreatment ANC <1.0×10⁹/L):
   at least 100% increase and an absolute increase >0.5×10⁹/L
   blast response (Bl) a ≥50% reduction in blast percentage in bone marrow and/or ≥50% reduction in peripheral blood total blast count.

Response Criteria for MDS. Response criteria are according to the International Working Group (Blood 2006; 108: 419-425). Responders are patients who obtain a CR, CRi, or PR, with or without cytogenetic response, hematologic improvements, and morphologic leukemia-free state.

A Morphologic Complete Response (CR) is defined as having peripheral blood count showing no circulating blasts, neutrophil count ≥1.0×10⁹/L, and platelet count ≥100×10⁹/L; and bone marrow aspirate and biopsy showing ≤5% blasts, and no extramedullary leukemia.

A Partial Response (PR) is defined as having all CR criteria if abnormal before treatment except ≥50% reduction in bone marrow blast but still >5%.

A Marrow CR is defined as having: in the bone marrow, ≤5% myeloblasts and decrease by 50% over pretreatment; and in peripheral blood, if HI responses, they are noted in addition to marrow CR.

Other criteria are as defined above.

Results. Results are given below.

Positive response after 1 cycle of treatment was observed in two out of two patients treated with Ca-EDTA chelation, antioxidants, minerals, and standard AML or MDS therapy, as follows.

Patient 1: A 75-80-year old female with heavily pre-treated metastatic stage IV ovarian cancer developed poor-risk therapy-related MDS. The MDS had adverse clinical features including a p53 mutation and monosomal karyotype with monosomy 7 (−7). She initiated standard MDS therapy comprising of decitabine. In addition, she received Ca-EDTA 0.75 g/m² combined with a daily mineral and antioxidant supplement, including zinc, selenium, magnesium, and vitamin C. After 1 cycle of therapy, her p53 mutation became undetectable. Additionally, her CA-125 level decreased from 832.9 to 803.3.

It was unexpected (in clinical practice of MDS and in the published literature of decitabine alone in AML/MDS) to observe a p53 mutation becoming undetectable during MDS therapy. For example, in New England Journal of Medicine, Welch et al. reported on cases of MDS/AML that achieved remission, but none showed a reduction in p53 mutation to undetectable levels. P53 protein mutation clearance was never complete in patients who had a response to decitabine, even in those with complete clinical remission (Welch et al., 2016).

Patient 2: A 65-70-year old female with AML with a normal karyotype and numerous molecular mutations (FLT3-ITD, IDH1, NRAS, U2AF1, DNMT3A, and BCOR) was treated. She received standard AML therapy comprising of decitabine×10 days, venetoclax, and as per standard of care, a FLT3-inihibitor (midostaurin) for the FLT3-ITD mutation. In addition, she received Ca-EDTA 0.75 g/m² combined with a daily mineral and antioxidant supplement, including zinc, selenium, magnesium, and vitamin C. After 1 cycle of treatment, her bone marrow blasts decreased significantly from 85% to 4% and the FLT3-ITD ratio decreased from 0.35 to 0.02. The patient demonstrated a complete bone marrow response, but with incomplete recovery of blood counts.

In addition, results from the study above yielded the responses shown in Table 11.

TABLE 11
Responses in Patients with AML and High-risk MDS Treated with Metal
Chelators Ca-EDTA or DMSA + Antioxidants/Minerals and Standard Therapy
(NCT03630991)

| | Number of Patients | |
|---|---|---|
| Disease Group | (Total n = 14) | Best Response |
| Front-line AML: De novo high- | n = 6 | 6/6 (100%) CR/CRi |
| risk AML (n = 1) and Secondary | | [CR (n = 5); CRi (n = 1)] |
| AML (therapy-related AML, | | |
| n = 2; evolved from prior heme | | |
| disorder, n = 3) | | |
| Refractory/Relapsed AML, | n = 2 | ½ (50%) CR |
| salvage 1 | | [CR (n = 1); No Response (n = 1)] |
| High-risk MDS/therapy-related | n = 5 | ⅘ (80%) CR [CR (n = 4); |
| MDS | | stable disease with TP53 mutation |
| | | undetectable after 1 cycle (n = 1)] |
| refractory MDS/MPN | n = 1 | CR (n = 1) |

Table Legend: *(⅚) front-line AML patients concurrently received the same hypomethylator-based therapy on a protocol of commercially available decitabine 10 days + commercially available venetoclax.

Most (5/6) front-line AML patients received the same hypomethylator-based therapy on a protocol of commercially available decitabine 10 days+commercially available venetoclax. Given that all 5 of these patients had either high-risk/unfavorable-risk AML or secondary AML, this CR/CRi rate of 100% is favorable.

The foregoing clinical trials of metal detoxification combined with standard therapy for MDS and AML suggest that this combination results in favorable overall responses, as well as, cytogenetic responses and molecular responses. These trials of metal detoxification+antioxidants/minerals+standard therapy in patients with AML and MDS demonstrate favorable clinical responses. For example, as shown in table 10, 100% of patients with de novo front-line AML (5/5) or relapsed/refractory AML in first salvage (4/4), who were treated with the metal chelator dexrazoxane+antioxidants/minerals (NCT03589729)+standard chemotherapy, attained complete remission (CR) or CR with incomplete neutrophil recovery (CRi) (Table 10).

### Metal Detoxification for High-Risk and Therapy-Related MDS.

As per Table 11, the CR rate of the 5 high-risk or therapy-related MDS patients who received standard of care hypomethylator based therapy concurrently with metal detoxification/rebalancing on protocol 2017-0752 (NCT03630991) with either DMSA or Ca-EDTA plus minerals/antioxidants (antioxidants/minerals shown in Table 10) was 4/5 (80%); one patient had stable disease (blasts dropped from 4% to 2%) along with the unexpected positive finding of the previously detected TP53 mutation becoming undetectable after 1 cycle. Four of these 5 high-risk or therapy-related MDS patients received standard of care decitabine ×3-5 days (n=4); 1 patient received standard-of-care azacytidine ×7 days +commercial idhifa). In the literature of hypomethylator-based therapy in higher-risk MDS, the overall response rate is 38-49%. Sekeres M A, et al. Randomized Phase II Study of Azacitidine Alone or in Combination With Lenalidomide or With Vorinostat in Higher-Risk Myelodysplastic Syndromes and Chronic Myelomonocytic Leukemia: North American Intergroup Study SWOG 51117.

Improvement in Cytogenetic and Molecular Profiles During Therapy. Among 8/10 AML or high-risk MDS patients undergoing standard front-line or salvage therapy+metal chelator dexrazoxane+antioxidants/minerals, the adverse cytogenetic or molecular abnormalities became undetectable (e.g. FLT3-ITD, t(3;3), RUNX1). On protocol 2017-0752 (NCT03630991), patients with high-risk MDS/AML, therapy-related MDS/AML, or relapsed/refractory MDS/AML, who received metal detoxification (Ca-EDTA or DMSA)+antioxidants/minerals+standard therapy (Table 1B), favorable responses were also observed. For example, all high-risk AML patients undergoing front-line AML therapy, including de novo high-risk AML (n=1) or secondary AML [n=5, (therapy-related AML, n=2; evolved from prior hematologic disorder, n=3)] achieved either CR (n=5) or CR with partial platelet recovery (CRi) (n=1). Furthermore, 4/5 high-risk MDS/therapy-related MDS patients achieved CR and 1 patient with refractory MDS/MPN achieved CR. In 4/5 evaluable patients with high-risk MDS/AML who received metal detoxification with Ca-EDTA or DMSA+antioxidants/minerals with standard therapy, adverse cytogenetic or molecular abnormalities became undetectable after treatment.

Reduction in the burden of mutated TP53. During therapy with either Ca-EDTA or DMSA combined with standard MDS/AML therapy, a reduction in TP53 mutation burden was observed. In 5 patients with MDS/AML, who possessed TP53 mutations at baseline, 100% demonstrated a reduction in the burden of mutated TP53. In 3/5 patients, the TP53 mutations became undetectable on next generation sequencing bone marrow samples collected after cycle 1; however, in two of those cases, the TP53 mutation was later re-detected, but only at a very low level (variant allelic frequency <2%).

### Comparison to Standards of Care

In comparison, based on the current literature of front-line AML, only approximately 60%-70% of adults with AML attain CR following standard induction therapy alone. See, e.g. Lambert J et al., Haematologica, 2019 January; 104(1):113-119. Complete remission rates of patients in salvage 1 patients are lower, estimated up to 50%. See, e.g., Megías-Vericat J E et al., Ann Hematol. 2018 July; 97(7):1115-1153) and Chevallier P et al., J Clin Oncol, 2008 Nov. 10; 26(32):5192-7.

Furthermore, unpublished data shows that CR/CRi rate of the 5 patients who concurrently received standard of care/commercially decitabine 10 days+venetoclax and metal detoxification/rebalancing on protocol (2017-0752)(NCT03630991) (denoted with asterisk below Table 11) was higher (100%) [CR (n=5)/CRi(n=1)] when compared to patients on the same protocol of standard of care/commercially available decitabine 10 days+venetoclax who did not receive metal detoxification (90%). Patients on the clinical trial of commercially available decitabine 10 days+venetoclax were allowed to be concurrently enrolled (if they met eligibility criteria) on the supportive care clinical trial (NCT03630991) of metal detoxification and rebalancing with either Ca-EDTA or DMSA+antioxidants/minerals. Among 48 front-line AML patients of all risk-types who did not enroll concurrently on the metal detoxification and rebalancing protocol, 43 (90%) achieved CR/CRi. Among 5 separate patients described in Table 11 who did receive metal detoxification and rebalancing concurrently with the decitabine 10 days+venetoclax protocol—all of whom had high-risk/secondary AML—all 5/5 (100%) achieved CR/CRi. The result is noteworthy considering that none of these 5 patients who received the concurrent metal detoxification and rebalancing regimen had favorable risk; in contrast, more of the patients on standard care, who did not receive the concurrent metal detoxification and rebalancing regimen, had a significantly more favorable risk: among these newly-diagnosed patients, 11 of these 48 patients were favorable-risk, the rest were intermediate-risk or adverse-risk AML.

A publication of standard of care hypomethylator therapy+venetoclax also showed lower CR/CRi rates than the 100% [CR (n=5)/CRi(n=1)] described above when metal rebalancing therapy was added. DiNardo CD et al., “Venetoclax combined with decitabine or azacitidine in treatment-naive, elderly patients with acute myeloid leukemia,” Blood, 2019 Jan. 3; 133(1):7-17, reported on patients (N=145) who were at least 65 years old with treatment-naive AML, among which 67% achieved CR+CRi.

Accordingly, chelation therapy combined with antioxidants, minerals, vitamins, and/or free radical scavenging agents may be used for detoxification and rebalancing of metals in leukemia patients receiving anti-cancer pharmacologic therapy.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Takenaga et al., J. Control Release, 52(1-2):81-87, 1998.
Remington's Pharmaceutical Sciences” 15^{th} Ed., 1035-1038 and 1570-1580, 1990.
Remington's Science and Practice of Pharmacy, 21^{st} Ed., Mack Printing Company, 2005.
Remington's Pharmaceutical Sciences, 18th Ed. Mack Printing Company, 1289-1329, 1990.
Mathiowitz et al., Nature, 386(6623):410-414, 1997.
Hwang et al., Crit. Rev. Ther. Drug Carrier Syst., 15(3):243-284, 1998.
U.S. Pat. Nos. 6,7537,514, 6,613,308, 5,466,468, 5,543,158; 5,641,515; and 5,399,363
VanLandingham et al., 2002; Ostrakhovitch and Cherian, 2005
Flora and Pachauri. Int J Environ Res Public Health. 2010;7(7):2745-2788.
FDA. DIMERCAPROL INJECTION, USP: BAL in Oil Ampules.
Lamas et al. Ann Intern Med. 2013;159(12):797-805.
Lamas et al. Am Heart J. 2014;168(1):37-44 e35.
Lamas et al. Jama. 2013;309(12):1241-1250.
99207-240-05 NCs, Corp PMP. CALCIUM DISODIUM VERSENATE-edetate calcium disodium injection: Package Insert 2013;
Bradberry et al. QJM. 2009;102(10):721-732.
Pharmaceuticals O. CHEMET® FDA Package Insert. 2007;
Bose-O'Reilly et al. Sci Total Environ. 2003;307(1-3):71-82.
List A F. Cancer Control. 2010;17 Supp1:2-8.
Cermak et al. Hemoglobin. 2011;35(3):217-227.
Cermak et al. Leuk Res. 2013;37(12):1612-1615.
Vachhani et al. Leuk Res Rep. 2017;7:36-39.
Schloemer et al. J Pediatr Hematol Oncol. 2017;39(5):e254-e258.
Walker et al. Pediatr Blood Cancer. 2013;60(4):616-620.
Woodlock et al. Am J Hematol. 1998;59(3):246-248.
FDA. ZINECARD® (dexrazoxane) for injection. 2014.
Fu et al. Mol Cancer Ther. 2012;11(6):1221-1225.
Chan et al. Clin Cancer Res. 2016.
FDA. Ethyol® (amifostine) for Injection, 2018.
Welch et al. N Engl J Med. 2016;375(21):2023-2036.
Frullano et al. Curr Org Synth. 8(4):535-565, 2011.
Koedrith P, Kim H, Weon J I, et al., Int J Hyg Environ Health 2013;216:587-598.
Garcia-Perez Jet al., Environmental research 2015;140:542-553.
Van Laecke S et al., Leukemia 2014;28:729-731.
Birt D F, Magnesium 1989;8:17-30.
Su Yet al., Clin Chim Acta 2011;412:2305-2309.
Wen C P et al., Cancer Res 2014;74:6589-6597.
Hong M, He G. 2016 J Transl Int Med 2017;5:69-71.
Huh Y O et al., American journal of clinical pathology 2001;116:437-443.
Ravandi F et al., Cancer 2017;123:426-435.
Gonzalez Garcia J R et al., Blood 2006;108:3952-3953; author reply 3953.
Poirel H et al., Blood 1996;87:2496-2505.
Boissel N et al., Blood 2005;106:3618-3620.
Boissel Net al., Leukemia 2002;16:1699-1704.
Preudhomme C et al., Blood 2002;100:2717-2723.
Wolff D J et al., J Mol Diagn 2007;9:134-143.
Dohner H et al., Blood 2017;129:424-447.
Trinidad AG et al., Mol Cell. 2013;50(6):805-817

1. A method of treating a proliferative disease in a mammalian subject comprising administering to the subject:
(i) one or more metal chelators;
(ii) an anti-cancer therapy; and
(iii) optionally, at least one antioxidant, vitamin, or essential mineral,
in a therapeutically effective amount.

2. The method of claim 1, wherein the antioxidant, vitamin(s) or mineral(s) is/are chosen from zinc, selenium, magnesium, rubidium and vitamin C.

3. The method of claim 2, wherein at least one of zinc, selenium, magnesium, and vitamin C are administered.

4. (canceled)

5. The method of claim 3, wherein zinc, selenium, magnesium, and vitamin C are administered.

6. The method of claim 1, wherein the one or more metal chelators are broad-spectrum metal chelators.

7. The method of claim 6, wherein at least one of the one or more metal chelators is/are capable of chelating at least two metals.

8. The method of claim 7, wherein the one or more metal chelators is/are administered in an amount effective to reduce the levels of the at least two metals.

9. (canceled)

10. (canceled)

11. The method of claim 1, wherein the one or more metal chelators are chosen from a dithiol chelator, an iron chelator, a copper chelator, and a gadolinium chelator.

12. The method of claim 1, wherein the one or more metal chelators are chosen from EDTA, dimercaptosuccinic acid (DMSA), 2,3-dimercapto-1-propanesulfonic acid (DMPS), N-(2,3-dimercaptopropyl)-phthalamidic acid (DMPA) dimercaprol (B AL), N-acetylcysteine (NAC), deferasirox, deferiprone, deferoxamine, pentetate calcium trisodium (Ca-DPTA), pentetate zinc trisodium (Zn-DPTA), trientine, tetrathiomolybdate, and dexrazoxane.

13. (canceled)

14. The method of claim 12, wherein the metal chelator is deferasirox, deferiprone, or deferoxamine.

15. The method of claim 11, wherein the gadolinium chelator is a bifunctional gadolinium(III) chelator.

16. (canceled)

17. The method of claim 11, wherein the copper chelator is trientine or tetrathiomolybdate.

18. The method of claim 12, wherein the metal chelator is dexrazoxane.

19. (canceled)

20. The method of claim 1, wherein the proliferative disease is chosen from cancer, a myeloproliferative neoplasm (MPN), myelodysplastic syndrome (MDS), bone marrow disease, a bone marrow failure, and a cytopenia.

21. (canceled)

22. The method of claim 20, wherein the cancer has relapsed or is refractory to a previous treatment.

23. The method of claim 20, wherein the cancer is a hematologic malignancy.

24. The method of claim 23, wherein the cancer is a leukemia.

25. The method of claim 24, wherein the leukemia is chosen from acute myeloid leukemia (AML), acute lymphoblastic leukemia (ALL), chronic lymphocytic leukemia (CLL), chronic myeloid leukemia (CML).

26. The method of claim 25, wherein the leukemia is AML.

27. (canceled)

28. (canceled)

29. (canceled)

30. (canceled)

31. (canceled)

32. (canceled)

33. The method of claim 1, wherein the anti-cancer therapy is chosen from a chemotherapy, an epigenetic therapy, an immunotherapy, or a targeted cancer therapy.

34. The method of claim 33, wherein the anti-cancer therapy is a chemotherapy.

35. The method of claim 34, wherein the chemotherapy comprises one or more agents chosen from mylotarg, cladribine, idarubicin, and cytarabine.

36. The method of claim 34, wherein the chemotherapy comprises cladribine, idarubicin, and cytarabine (“CLIA”).

37. The method of claim 34, wherein the chemotherapy comprises mylotarg, cladribine, idarubicin, and cytarabine (“CLIA-M”).

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. The method of claim 1, wherein the human subject:
a) has a cancer; and
b) has elevated levels of one or more metals as compared to healthy subjects.

50. The method of claim 49, wherein the elevated levels of one or more metals are measured in the bone marrow and/or the serum.

51. (canceled)

52. (canceled)

53. The method of claim 50, wherein the metal(s) is/are chosen from chosen from arsenic (As), aluminum (Al), antimony (Sb), Barium (B a), boron (B), cadmium (Cd), Cerium (Ce), Chromium (Cr), lead (Pb), mercury (Hg), neodymium (Nd), Nickel (Ni), tin (Sn), titanium (Ti), uranium (U), vanadium (V), copper (Cu), and iron (Fe).

54. The method of claim 49, wherein the human subject has decreased levels of at least one of calcium (Ca), magnesium (Mg), selenium (Se), zinc (Zn) and rubidium (Rb).

55. The method of claim 54, wherein the elevated and/or reduced levels are with respect to the median values in a non-diseased population.

56. The method of claim 1, wherein the method results in the reduction or clearance of one or more mutations or cytogenetic abnormalities in the proliferative disease.

57. (canceled)

58. A method of diagnosing a subject with a chelation therapy-responsive proliferative disease, comprising:
i) measuring the levels of two or more metals in one or more samples of the subject's serum or bone marrow;
ii) comparing the levels of each of the two or more metals in the sample(s) to each of two or more corresponding median reference values of the same metals obtained from healthy patients; and
iii) if the levels of the two or more metals in the sample(s) are higher than the median reference values, classifying the subject as having a chelation therapy-responsive proliferative disease.

59. The method of claim 58, further comprising
treating the diagnosed chelation therapy-responsive disease by administering to the subject:
(a) one or more metal chelators; and
(b) at least one of zinc, selenium, magnesium, rubidium and/or vitamin C, in a therapeutically effective amount.

60. A pharmaceutical composition or pharmaceutical combination comprising
(i) one or more metal chelators;
(ii) at least one antioxidant, vitamin, or essential mineral; and
(iii) a pharmaceutically acceptable excipient.

61. (canceled)

62. (canceled)

63. (canceled)

64. (canceled)

65. (canceled)

66. (canceled)

67. (canceled)

68. (canceled)

69. (canceled)
